(19) United States
(12) Patent Application Publication
(10) Pub. No.: US 2005/0147664 A1
(43) Pub. Date: Jul. 7, 2005

(54) Compositions comprising antibodies and methods of using the same for targeting nanoparticulate active agent delivery

(75) Inventors: Elaine Liversidge, West Chester, PA (US); James Cunningham, Malvern, PA (US)

Correspondence Address: FOLEY AND LARDNER;SUITE 500, 3000 K STREET NW, WASHINGTON, DC, 20007, US

(73) Assignee: Elan Pharma International Ltd.

(21) Appl. No.: 10/979,792

(22) Filed: Nov. 3, 2004

Related U.S. Application Data
(60) Provisional application No. 60/519,251, filed on Nov. 13, 2003.

(51) Int. Cl. A61K 9/48; A61K 9/26; A61K 9/14

(52) U.S. Cl. 424/452; 424/469; 424/489

(57) ABSTRACT

The present invention is directed to compositions of one or more nanoparticulate active agents, at least one PEG-derivatized surface stabilizer, and at least one antibody or fragment thereof, and methods of using such compositions for targeting delivery of the one or more active agents to a desired site. The one or more active agents preferably have a particle size of about 2 microns or less. The targeted delivery can be used, for example, for disease sensing, imaging, or drug delivery.

### FIELD OF THE INVENTION

The present invention is directed to compositions of one or more nanoparticulate active agents, at least one PEG-derivatized surface stabilizer, and at least one antibody or fragment thereof. Also encompassed by the invention are methods of using such compositions for targeting delivery of the one or more active agents to a desired site. The one or more active agents preferably have a particle size of less than about 2 microns. The targeted delivery can be used, for example, for disease sensing, imaging, or drug delivery.

### BACKGROUND OF THE INVENTION

I. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed on or associated with the surface thereof a non-crosslinked surface stabilizer. This invention is an improvement over that disclosed in the '684 patent, as the '684 patent does not describe compositions comprising an antibody or an antibody fragment.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described in, for example, U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using lodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate,” 6,428,814 for “Bioadhesive Nanoparticulate Compositions Having Cationic Surface Stabilizers;” 6,431,478 for “Small Scale Mill;” 6,432,381 for “Methods for Targeting Drug Delivery to the Upper and/or Lower Gastrointestinal Tract,” 6,592,903 for “Nanoparticulate dispersions comprising a synergistic combination of a polymeric surface stabilizer and dioctyl sodium Sulfosuccinate;” and 6,582,285 for “Apparatus for sanitary wet milling;” all of which are specifically incorporated by reference. In addition, U.S. patent application No. 20020012675 A1, published on Jan. 31, 2002, for “Controlled Release Nanoparticulate Compositions,” and WO 02/098565 for “System and Method for Milling Materials,” describe nanoparticulate active agent compositions, and are specifically incorporated by reference. None of these references describe nanoparticulate active agent compositions comprising an antibody or a fragment thereof.

Amorphous small particle compositions are described in, for example, U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

II. Background Regarding Antibodies

Antibodies are one of two important antigen recognition molecules of the immune system. The other antigen recognition molecule is found on the T cell, called the T cell receptor (TcR). There are five different classes of antibodies or immunoglobulins (Ig) known as IgD, IgA, IgM, IgE, and IgG. There are four subclasses of IgG and two subclasses of IgA.

Antibodies recognize antigen. An antigen is a substance capable of inducing a specific immune response. See http://www.medicine.dal.ca/micro/education/pimunit/atb.htm.

Antibodies, as exemplified by IgG, are Y-shaped proteins composed of two identical heavy chains and two identical light chains joined by disulfide linkages. The heavy chain is the largest (and thus the heaviest). Two types of light chains exist (kappa or lambda) and each antibody has one or the other type. The heavy and light chains are folded into domains and are held together by disulphide bridges.

The two most important regions of the antibody are: (1) the Fab region and (2) the Fc region. For the Fab region, the ‘F’ stands for fragment and the ‘ab’ stands for antigen binding. This is the region where antigen binding takes place. The Fab portion of an antibody is made up of the N-terminal domains of both the heavy and light chains. This domain of the antibody is called the variable (V) domain (VH on the heavy chain, VL on the light chain) because there is such high variability between antibodies in this area. The high variability allows the recognition of thousands of different antigens.

The Fc portion of the antibody is limited in variability and is responsible for the biological activity of the antibody (as opposed to antigen binding). The Fc portion varies between antibody classes (and subclasses) but is identical within that class. The C-terminal end of the heavy chains form the Fc region. The Fc region plays an important role in receptor binding.

Each part of the antigen that is recognized by an antibody is known as an epitope. Each epitope of an antigen binds to one of the Fab regions of antibodies. The interaction between the two proteins involves noncovalent bonding forces. The binding, therefore, is reversible and the strength of the interaction depends on how well the antigen and the antibody. “fit” together, as well as if there is a second epitope for the other Fab to bind.

As noted above, there are five different classes of antibodies or immunoglobulin, including IgA, IgM, IgD, IgE, and IgG. IgA is found in low levels (concentration is usually around 3.5 mg/ml) in the monomeric form in circulation, but it is most common and most active at mucosal surfaces, where it appears as a dimeric protein. The dimeric IgA provides the primary defense at mucosal surfaces such as bronchioles, nasal mucosa, prostate, vagina, and intestine. IgA is also abundant in saliva, tears and breast milk, especially colostrum.

IgM can be found as “surface antibody” on the surface membrane of B cells (sIgM) or as a 5-subunit macromolecule secreted into the blood by plasma cells. The surface IgM is structurally different in the Fc region from the secreted form since it must bind through the membrane. Surface IgM binds directly as an integral membrane protein, it does not bind to an IgM Fc receptor like IgE does. Secreted IgM is found as a “pentameric” molecule. The five IgM subunits are held together providing multiple binding sites in each molecule. Pentameric IgM binds to antigens on the surface of a bacteria like a spider.

IgD is found on thesurface of most B lymphocytes just like sIgM. So far, the function of this antibody is unknown but it has been suggested that it acts as an antigen receptor and that it is needed for B cell activation. A very small amount of IgD is secreted, and its functions as a secreted antibody are unclear.

Like IgA, IgE is particularly effective at mucosal surfaces. It is also active in the blood and in the tissues. The serum concentration of this antibody is normally very low as most IgE is tightly bound to its receptors on mast cells and basophils. The production of IgE is controlled by cytokines and this class is responsible for Type I hypersentitivity reactions (allergic and anaphylactic). IgE is found to increase greatly in response to parasitic infection.

IgG is the most abundant class of antibody in the blood (serum concentration is 13 mg/ml). There are four subclasses of IgG which are all monomeric and they usually have a very high affinity for antigen. Unlike IgM, IgG is able to leave the blood stream and enter tissues. IgG is also the only class of antibody to pass the placental barrier. Therefore IgG provides the only antibody protection for newborns until their own immune system is able to contribute to antibody production. The subclasses of antibody IgG produced is dependant on the cytokines present (especially IL-4 and IL-2) and each class has its own special activity. IgG also plays an important role in neutralizing toxins (from bacterial infection for example) in the blood and tissues. See http://www.medicine.dal.ca/micro/education/pimunit/atb.htm.

Because antibody molecules very precisely “recognize” and bind to certain shapes on other molecules, they can be used as targeting tools. See e.g., “The Study of Antibody Recognition©” http://www.antibodyresource.com/.

To distinguish between both self and a multitude of foreign species, antibodies need to have a highly discriminating method of recognition on the molecular level. This specificity is the result of the complementary nature of antibody binding. This characteristic of antibody binding is the result of immunologically-tuned interactions (i.e., charge-charge, dipole-dipole, H-bonding, and Van der Waals) between the antigen and amino acid residues present in the antibody binding pocket. By taking advantage of the varied chemical properties of the 20 amino acids, the immune system is able to generate an array of antibody binding pockets that can accommodate the shape, charge, and hydrophobicity of seemingly any given antigen. The complementary nature of antibody binding has been confirmed with the aid of X-ray crystallography.

The high degree of complementarity exhibited by antibody binding also endows antibodies with high affinities for their antigens. For a mature immune response, antibody affinities typically fall in the range of 10⁵ to 10¹² M⁻¹. Recently, an upper ceiling for the affinity of a “normal” immune response has been proposed to be approximately 10¹⁰ M⁻¹.

Two types of antibody samples are known. The first type, polyclonal antibodies, can be obtained by immunizing a mammal, such as a goat, sheep, mouse, or, most conveniently, a rabbit. After immunization, blood is removed (periodically, if desired) and the antibodies can be purified directly from the serum. Polyclonal antibodies originate from a variety of B-cells that differ in the genetic material that encodes for antibody production. In a polyclonal sample, some of the antibodies will be specific for the antigen with which the animal was immunized. The remaining antibodies have been elicited from encounters with other foreign antigens that the animal has been exposed to throughout its lifetime.

The second type of antibody sample, the monoclonal antibody, is derived from a more complex process. Here, a mammal, almost always an inbred mouse, is immunized with an antigen. After repeated immunizations, the spleen of the animal is removed. Because the spleen is responsible for B-cell production, the spleen cells contain the genetic information that gives rise to antibody production. Unfortunately, these spleen cells cannot be cultured. As a result, they are fused with “immortal” myeloma cells, so-called because of their ability to proliferate in vitro. The resulting fused cells, called hybridoma cells, are screened with a suitable immunoassay, such as a colorometric enzyme-linked immunoabsorbant assay (ELISA). Use of this assay allows for the selection of hybridoma cells that produce antigen-specific antibodies. Because a given hybridoma cell is derived from a single B-cell, it produces a monoclonal antibody. Once a single hybridoma line is selected, it is injected into a healthy mouse. Hybridoma cells, like myeloma cells, have the ability to produce tumors; consequently, after injection with a hybridoma line, a tumor grows inside the host mouse. When this tumor grows, it produces ascites, a fluid that is rich in monoclonal antibodies.

Both polyclonal and monoclonal antibodies offer certain advantages. Polyclonal antibodies are inexpensive to produce relative to the cost of monoclonal antibody technology. In addition, large quantities of polyclonal antibodies (˜10 mg/mL) can be produced from the serum of an immunized animal. Finally, high affinity polyclonal antibodies can be isolated merely 2-3 months after the initial immunization.

However, monoclonal antibodies have certain advantages over polyclonal antibodies. Because of their immortal nature, hybridoma cells can be frozen, thawed, and recultured in vitro. As a result, for a given monoclonal line, there exists a constant and renewable source of antibodies for. study. In addition, the defined composition of a monoclonal antibody allows for its chemical composition, on a molecular level, to be analyzed in detail. For example, X-ray crystallographic and gene sequencing methods can only be applied to monoclonal antibodies. This level of detail is particularly useful when studying mechanistic issues related to binding.

To play their physiological roles, antibodies are required to exhibit exquisite specificity and high affinity for antigens. These properties enable the use of antibodies as targeting tools.

III. Background Regarding PEG-Derivatized Surface Stabilizers

Polyethylene glycol (PEG) derivatized surface stabilizers for nanoparticulate active agents were first described in U.S. Pat. No. 6,270,806, which is specifically incorporated by reference. Examples of such surface stabilizers are PEG-derivatized phospholipid, PEG-derivatized cholesterol, PEG-derivatized cholesterol derivative, PEG-derivatized vitamin A, or PEG-derivatized vitamin E surface stabilizers.

PEG-derivatized lipids are described in, for example, U.S. Pat. No. 5,672,662 (“the '662 Patent”) for “Poly(Ethylene Glycol) and Related Polymers Monosubstituted with Propionic or Butanoic Acids and Functional Derivatives Thereof for Biotechnical Applications,” and Yuda et al., “Prolongation of Liposome Circulation Time by Various Derivatives of Polyethyleneglycols,” Biol. Pharm. Bull., 19:1347-1351, 1347-1348, 1349 (1996).

PEG-derivatized surface stabilizers can be desirable for several reasons. For example, PEG-Derivatized surface stabilizers can result in increased circulation time for the component nanoparticulate active agent. In addition, PEG-Derivatized surface stabilizers can result in decreased toxicity of the component nanoparticulate active agent. Moreover, PEG-Derivatized surface stabilizers can result in increased stability of the component nanoparticulate active agent.

IV. Background Regarding Targeted Drug Delivery

Advances in the development of novel therapeutic molecules have exceeded advances in the development of delivery technologies to enable the therapeutic use of those molecules. For example, antisense oligonucleotides, active only in the nucleus, have enormous potential; however their very poor trafficking into the nuclear compartment has rendered them of very little value. Other examples include plasmid DNA, transcription factors and antibodies against intracellular targets. Thus, targeting of therapeutics to particular subcellular locations is an important challenge. Even the challenge of delivery of macromolecular drugs to specific locations in the body, such as cancer metastases or sites of inflammation, represents an unmet and substantial challenge.

In addition to increasing the usefulness of new drugs, targeted delivery can also decrease the toxicity of known drugs. See Pasqualini et al., “Cancer treatment by targeted drug delivery to tumor vasculature in a mouse model,” Science, 279:323-4 (Jan. 16, 1998).

Successful drug targeting is a very complicated problem. It involves affecting the various distributional and rate processes, as well as sometimes the drug metabolism and disposition. Factors to be considered in designing drug targeting include the nature of biological and cellular membranes, distribution and presence of drug receptors, as well as the enzymes responsible for drug metabolism, time-plasma concentration profiles, and local blood flow. See N. Bodor, “Retrometabolic Approaches to Drug Targeting, pp. 1-27, in Membranes and Barriers: Targeted Drug Delivery, NIDA Research Monograph, Number 154 (1995).

One way of aiding this process is to physically apply the drug to the target site of interest. For example, Durect Corporation is developing proprietary miniaturized catheter technology that can be used to direct the flow of a drug to the target organ, tissue or synthetic medical structure, such as a graft. Site-specific delivery enables a therapeutic concentration of a drug to be administered to the desired target without exposing the entire body to a similar dose. See http://www.durect.com/wt/durect/page_name/delivery. A disadvantage to this method is it is not always feasible to target a specific cell type, such as a mucous cell, cancer cell, epithelial cell, etc. with precision using such technology.

Self assembly of amphiphilic components has been explored for many years in drug delivery, the most notable example being development of liposome carriers. This challenge has been rather frustrating, in that the inherent instability of these systems has rendered them also of relatively little value. Rapid removal of the liposomes from the systemic circulation by the reticuloendothelial system of the liver has been addressed by PEGylation of the phospholipid components, however this has created a quandary: non-PEGylated lipid formulations are rapidly cleared from the circulation, and PEGylation to adequate extents dramatically decreases the stability of the supermolecular assembly. See http://www.biomed.mat.ethz.ch/research/res_topics/Project3.

Other approaches to drug targeting include retrometabolic approaches, See N. Bodor, “Retrometabolic Approaches to Drug Targeting, pp. 1-27, in Membranes and Barriers: Targeted Drug Delivery, NIDA Research Monograph, Number 154 (1995).

Yet other methods employ designing a drug which, as part of its structure, inherently targets a desired site. See e.g., Davis et al., “Conformationally Constrained Peptide Drugs Targeted at the Blood-Brain Barrier,” pp 47-60, in Membranes and Barriers: Targeted Drug Delivery, NIDA Research Monograph, Number 154 (1995). A problem with this approach is that it cannot be used to improve drug delivery for conventional existing drugs. Moreover, designing such drugs can be costly and time consuming.

Another example of targeted drug delivery is described by Chourasia et al., “Pharmaceutical Approaches to Colon Targeted Drug Delivery Systems,” J. Pharm. Pharmaceut. Sci., 6(1):33-66 ( 2003). This reference notes that although oral delivery has become a widely accepted route of administration of therapeutic drugs, the gastrointestinal tract presents several formidable barriers to drug delivery. Colonic drug delivery has gained increased importance not just for the delivery of the drugs for the treatment of local diseases associated with the colon, such as Crohn's diseases, ulcerative colitis, colorectal cancer and amebiasis, but also for its potential for the delivery of proteins and therapeutic peptides.

The various strategies for targeting orally administered drugs to the colon include covalent linkage of a drug with a carrier, coating with pH-sensitive polymers, formulation of timed released systems, exploitation of carriers that are degraded specifically by colonic bacteria, bioadhesive systems and osmotic controlled drug delivery systems.

Covalent linkage of a drug with a carrier can change the binding or biological properties of a drug, and therefore can be undesirable. Moreover, none of these approaches are applicable to virtually any drug, to be targeted to any desired site.

There is a need in the art for methods of targeting active agent delivery having greater selectively, localized active agent delivery, and effectiveness than that observed with conventional active agent solution or microparticulate formulations or prior art nanoparticulate active agent formulations. The present invention satisfies these needs.

### SUMMARY OF THE INVENTION

The present invention is directed to the surprising and unexpected discovery that compositions comprising one or more nanoparticulate active agents, preferably having an effective average particle size of less than about 2 microns, can be made utilizing at least one antibody or a fragment thereof. The compositions are useful in targeting delivery of the one or more active agents to a desired target site. The targeted delivery can be used, for example, for disease sensing, imaging, or drug delivery.

One embodiment of the invention is directed to compositions comprising: (1) one or more active agents, preferably having a particle size of less than about two microns; (2) at least one PEG-derivatized surface stabilizer adsorbed to or associated with the surface of the one or more active agents; and (3) at least one antibody, or a fragment thereof, which is associated, either directly or indirectly, with the at least one PEG-derivatized surface stabilizer. The antibody or fragment thereof specifically binds to a target site of interest. Preferably, the one or more active agents are poorly soluble in at least one liquid media.

The invention also encompasses pharmaceutical compositions comprising a composition of the invention. The pharmaceutical compositions preferably comprise: (1) at least one active agent, preferably having a particle size of less than about two microns; (2) at least one PEG-derivatized surface stabilizer adsorbed on or associated with the surface of the active agent; (3) at least one antibody, or a fragment thereof, which is associated, either directly or indirectly, with the at least one PEG-derivatized surface stabilizer; and (4) at least one pharmaceutically acceptable carrier, as well as any desired excipients.

This invention further discloses methods of making the compositions of the invention. Such a method comprises contacting an active agent with at least one PEG-derivatized surface stabilizer and at least one antibody or a fragment thereof for a time and under conditions sufficient to provide an active agent/PEG-derivatized surface stabilizer/antibody composition. The resultant active agent preferably has a particle size of less than about 2 microns. The at least one PEG-derivatized surface stabilizer and at least one antibody or fragment thereof can be contacted with the active agent either before, during, or after size reduction of the active agent. Preferably, the PEG-derivatized surface stabilizer is contacted with the active agent during size reduction of the active agent, and then subsequently the antibody or fragment thereof is contacted with the active agent/PEG-derivatized surface stabilizer composition.

Finally, the invention encompasses methods of targeted active agent delivery utilizing the compositions of the invention. In such a method, the antibody or a fragment thereof present in the compositions of the invention specifically targets or binds to a site of interest. This results in the active agent present in the compositions of the invention being delivered to the target site of interest. The antibody, or fragment thereof having binding ability, can be chosen to bind to any site of interest. This method can result in more effective compositions, as well as the need to administer active agents in smaller doses.

Both the foregoing general description and the following brief description of the drawings and detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1A: Shows the active formulation incubated with Avidin-FITC (phase contrast);

FIG. 1B: Shows the active formulation incubated with Avidin-FITC (epifluorescence);

FIG. 1C: Shows the control formulation incubated with Avidin-FITC (phase contrast);

FIG. 1D: Shows the control formulation incubated with Avidin-FITC (epifluorescence);

FIG. 2A: Shows fluorescent WIN 68209 dispersion imaged by phase contrast;

FIG. 2B: Shows fluorescent WIN 68209 dispersion imaged by epifluorescence;

FIG. 3A: Shows anti-integrin αᵥβ₃-coated microspheres binding to HUVEC;

FIG. 3B: Shows anti-integrin αᵥβ₃-coated microspheres failing to bind to NIH 3T3 Fibroblasts;

FIG. 4A: Shows antibody-decorated nanoparticulate WIN 68209 particles in the active sample bound strongly to HLTVEC as demonstrated by epifluorescence;

FIG. 4B: Shows antibody-decorated nanoparticulate WIN 68209 particles in the active sample bound strongly to HUVEC as demonstrated by phase contrast;

FIG. 4C: Shows the failure of nanoparticulate WIN 68209 particles in the control sample to strongly bind to HUVEC as demonstrated by epifluorescence; and

FIG. 4D: Shows the failure of nanoparticulate WIN 68209 particles in the control sample to strongly bind to HUVEC as demonstrated by phase contrast.

### DETAILED DESCRIPTION OF THE INVENTION

The present invention is directed to the surprising and unexpected discovery that compositions comprising one or more active agents, preferably having a particle size of less than about 2 microns, can be made utilizing at least one antibody or a fragment thereof. The compositions are useful in targeting delivery of the one or more active agents to a desired site. The compositions and methods can result in dramatically superior bioavailability, decreased toxicity and undesirable side effects, fast onset of therapeutic activity, and effectiveness as compared to prior art active agent compositions.

The targeted delivery can be used, for example, for disease sensing, imaging, or drug delivery. For example, Akerman et al., “Nanocrystal Targeting In Vivo,” PNAS, 12617-12621 (Oct. 1, 2002), describes disease sensing and targeting utilizing semiconductor quantum dots (qdots) having attached thereto peptides which target specific vascular markers. This reference does not teach or suggest the use of nanoparticulate active agents, nor antibodies as targeting tools.

The active agent delivery systems of the invention provide a unique opportunity to selectively partition high concentrations of active agent to diseased biological surfaces or a target site of interest. By optimizing active agent exposure to the diseased or target site, the therapeutic or diagnostic potential can be dramatically enhanced.

The compositions of the invention comprises: (1) one or more active agents, preferably having a particle size of less than about two microns, and preferably poorly soluble in at least one liquid media; (2) at least one PEG-derivatized surface stabilizer adsorbed to or associated with the surface of the one or more active agents; and (3) at least one antibody, or a fragment thereof, which is associated either directly or indirectly via for example a linker, with the at least one PEG-derivatized surface stabilizer. The antibody or fragment thereof specifically binds to a target site of interest.

An antibody or fragment thereof can be attached to a PEG-derivatized surface stabilizer directly or indirectly using a suitable attachment mechanism. Direct linkage can be accomplished by, for example, covalently binding the antibody or fragment thereof to the PEG-derivatized surface stabilizer. Indirect linkage can be accomplished using, for example, a multivalent adapter element, or via other non-covalent coupling. The most common multivalent adapter elements are biotin and streptavidin.

The present invention also includes the active agent compositions of the invention together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid: or aerosol form, vaginal, nasal, rectal, ocular, local (powders, ointments or drops), buccal, intracisternal, intraperitoneal, or topical administration, and the like.

It was unexpectedly discovered that in the compositions of the invention, the antibody or fragment thereof retains its binding ability, required for targeting, following attachment to the PEG-derivatized surface stabilizer. The targeting aspect of the present invention is dependent upon the ability of the antibody or fragment thereof present in the compositions of the invention to bind to a target site of interest. If attachment of the antibody or fragment thereof, either directly or indirectly, to a PEG-derivatized surface stabilizer altered the ability of the antibody or fragment thereof to bind to a target site of interest, then the usefulness of the compositions of the invention would be dramatically diminished or lost.

Antibodies and fragments thereof noncovalently bind to a target site in a “lock and key” mechanism in which the antibody, or fragment thereof having binding capacity, has a three dimensional structure such that it binds to a target site having a complementary structure. Thus, a modification in the three dimensional structure of an antibody, or a fragment thereof having binding capacity, could destroy the antibody's ability to bind specifically to the target site.

Moreover, it was also surprisingly discovered that in the compositions of the invention, the PEG-derivatized surface stabilizer fimctions as desired following attachment of an antibody or fragment thereof to the surface stabilizer. As discussed extensively in the prior literature, and in particular in U.S. Pat. No. 6,270,806, the PEG-derivatized surface stabilizer adsorbs to or associates with the surface of the active agent and functions to “stabilize” the active agent against particle size growth. Particle size growth can occur by active agent agglomeration and by solubilization and subsequent recrystallization of the active agent. The PEG-derivatized surface stabilizer prevents these events from occurring, thereby maintaining the chemical and physical stability of the active agent. It was surprisingly discovered that the attachment of an antibody or fragment thereof to the PEG-derivatized surface stabilizer does not alter the ability of the surface stabilizer to stabilize the active agent. This is significant as if the PEG-derivatized surface stabilizer, following attachment of the antibody or fragment thereof, no longer functioned to prevent particle size growth of the active agent, then the active agent composition would lose the benefits accorded by being formulated into a small particle composition.

Finally, it was also surprisingly discovered that the active agent retains its activity following attachment of the PEG-derivatized surface stabilizer having an antibody or fragment thereof attached thereto.

Benefits of the compositions of the invention as compared to prior compositions of the same active agent include, but are not limited to: (1) dramatically improved active agent targeting; (2) increased bioavailability; (3) decreased toxicity; (4) smaller doses of active agent required to obtain the same pharmacological effect; (5) smaller tablet or other solid dosage form size or smaller liquid dose volumes; (6) faster onset of action; (7) a potential decrease in the frequency of dosing; (8) substantially similar or bioequivalent pharmacokinetic profiles of the active agent compositions when administered in the fed versus the fasted state; (9) an increased rate of dissolution for the active agent compositions; (10) high redispersibility of the active agent particles present in the compositions of the invention following administration; (11) improved performance characteristics for oral, intravenous, subcutaneous, or intramuscular injection, such as higher dose loading; (12) improved pharmacokinetic profiles, such as improved Tₘₐₓ, Cₘₐₓ, and AUC profiles; (13) low viscosity liquid active agent dosage forms can be made; (14) for liquid active agent compositions having a low viscosity—better subject compliance due to the perception of a lighter formulation which is easier to consume and digest; (15) for liquid active agent compositions having a low viscosity—ease of dispensing because one can use a cup or a syringe; and (16) the active agent compositions do not require organic solvents or pH extremes.

The present invention is described herein using several definitions that are set forth below and throughout the specification.

“About” will be understood by persons of ordinary skill in the art and will vary to some extent on the context in which the term is used. If there are uses of the term that are not clear to persons of ordinary skill in the art given the context in which it is used, “about” will mean up to plus or minus 10% of the particular term.

“Conventional” or “non-nanoparticulate active agent” means an active agent that is solubilized or that has an effective average particle size of greater than about 2 microns. “Effective average particle size of greater than about 2 microns” means that at least 50% of the particles of the composition have a size greater than about 2 microns.

As used herein, “nanoparticulate” refers to particulate active agent compositions having an effective average particle size of less than about 2 microns. “Effective average particle size of less than about 2 microns” means that at least 50% of the particles of the composition have a size less than about 2 microns.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such aslamines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Poorly water soluble active agents” as used herein means active agents having a solubility of less than about 30 mg/ml, preferably less than about 20 mg/ml, preferably less than about 10 mg/ml, or preferably less than about 1 mg/ml. Such active agents tend to be eliminated from the gastrointestinal tract before being absorbed into the circulation. Moreover, poorly water soluble active agents tend to be unsafe for intravenous administration techniques, which are used primarily in conjunction with highly water soluble active agents.

As used herein with reference to stable active agent particles, “stable” includes, but is not limited to, one or more of the following parameters: (1) that the active agent particles do not appreciably flocculate or agglomerate due to interparticle attractive forces, or otherwise significantly increase in particle size over time; (2) that the physical structure of the active agent particles is not altered over time, such as by conversion from an amorphous phase to crystalline phase; (3) that the active agent particles are chemically stable; and/or (4) where the active agent has not been subject to a heating step at or above the melting point of the active agent in the preparation of the compositions of the invention.

“Therapeutically effective amount” as used herein with respect to an active agent dosage, means a dosage that provides the specific pharmacological response for which the active agent is administered in a significant number of subjects in need of such treatment. A “therapeutically effective amount,” administered to a particular subject in a particular instance, will not always effectively treat the diseases described herein, even though such dosage is deemed a ‘therapeutically effective amount’ by those skilled in the art. Throughout this description, active agent dosages are, in particular instances, measured as oral dosages, or with reference to active agent levels as measured in blood.

I. Preferred Characteristics of the Active Agent Compositions of the Invention

A. Active Agent Targeting

This invention enables the targeting of active agents to specific sites. Targeting is accomplished by binding or attaching, either directly or indirectly, at least one antibody or a fragment thereof, which has the ability to selectively bind to a target site, to a PEG-derivatized surface stabilizer. The PEG-derivatized surface stabilizer is adsorbed or associated with the surface of an active agent. As described in more detail below, the PEG-derivatized surface stabilizer is not covalently bound to the active agent.

The PEG-derivatized surface stabilizer is an optimal tool for combining the small particle active agent and an antibody or fragment thereof, as the PEG derivitization provides an active site for binding directly to an antibody, or for binding to a linker which connects the antibody or fragment thereof and surface stabilizer.

1. Biotinylated Antibodies

In the first example described herein, a biotinylated monoclonal antibody is coupled indirectly to a biotinylated PEG-derivatized surface stabilizer using the protein avidin as a linker. A wide range of biotinylated monoclonal antibodies are available for different biological targets. Applications may range from topical formulations to tumor-targeted injectable formulations of anticancer agents, such as paclitaxel.

The antibody used in the example below binds selectively to integrin αᵥβ₃, which is a cell-adhesion receptor expressed on endothelial cells during angiogenesis.

2. Exemplary Targets, Diseases, and/or Conditions to be Treated

a. Esophageal Epithelial Cells

Examples of diseases of the esophagus that could be treated with the compositions of the invention include esophageal cancer and acid reflux disease.

Each year approximately 200,000 new cases of gastrointestinal malignancy are diagnosed in the United States. Most of these malignancies will be colorectal tumors, but cancer of the esophagus is on the rise and accounts for about 5% of all gastrointestinal malignancies. Due to the high incidence, colorectal cancer has been a primary target for research and development efforts to improve diagnosis and treatment for this disease. However, very little has been done to improve the poor prognosis confronted by the population suffering from esophageal cancer.

Esophageal cancer, with an estimated 12,300 new cases each year, is relatively uncommon in the United States, but the morbidity rate associated with the disease is extremely high. Approximately 98% of diagnosed patients will succumb to the disease, and the incidence of the disease, for still unknown reasons, is increasing at an alarming rate. As in many cancerous conditions, risk factors associated with the disease are multifaceted: age, diet, genetics, and environmental exposure have all been implicated in the etiology of the disease. In particular, the disease has been associated with patients being treating for gastroesophageal reflux and lung cancer. In addition, there have been numerous reports associating esophageal adenocarcinomas with a pre-cancerous condition known as Barrett's Esophagus. Barrett's Esophagus is diagnosed by evidence of metaplasia of the epithelial lining of the distal esophagus resulting from the chronic mucosal injury experienced by 10% of patients with long-lasting gastroesophgeal reflux disease. It is believed that the constant exposure of the epithelial lining in the distal portion of the esophagus to an acidic environment, with time, induces epithelial cell transformation.

One of the primary problems in treating esophageal cancer is that all current chemotherapeutic/chemopreventive agents have been formulated to maximize delivery to the stomach and lower gut regions. Alternatively, the oral cavity is treated locally using agents formulated as a mouth rinse. The esophagus, the about nine inch muscular tube connecting the oral cavity with the stomach, is by-passed in both administration regimens.

An active agent composition according to the invention for delivery targeted to the surface of esophageal epithelial cells can comprise: (1) at least one active agent useful in treating the esophageal condition or disease; (2) at least one PEG-derivatized surface stabilizer; and (3) at least one antibody or fragment thereof that specifically binds to the targeted epithelial cell(s).

If such a composition is to be used for treatment of esophageal cancer, the composition can comprise, for example, at least one anticancer active agent. Additional non-PEG-derivatized and PEG-derivatized surface stabilizers, excipients, and carriers can also be employed in the composition. Examples of anticancer agents are provided in U.S. Pat. Nos. 5,399,363 and 5,494,683, both for “Surface Modified Anticancer Nanoparticles,” which are specifically incorporated by reference.

b. Gastroesophageal Reflux Disease (GERD)

Evidence indicates that up to 44% of otherwise healthy adult Americans suffer from heartburn at least once a month. Approximately 7% of the population experience heartburn as often as once a day. It has been estimated that approximately 2% of the adult population suffers from Gastroesophageal reflux disease (GERD), based on objective measures such as endoscopic or histological examinations. The incidence of GERD increases markedly after the age of 40, and it is not uncommon for patients experiencing symptoms to wait years before seeking medical treatment.

Almost everyone experiences a little acid reflux, particularly after meals. Acid reflux irritates the walls of the esophagus, inducing a secondary peristalic contraction of the smooth muscle, and may produce the discomfort or pain known as heartburn.

After a meal, the lower esophageal sphincter (LES) usually remains closed. When it relaxes at an inappropriate time, it allows acid and food particles to reflux into the esophagus. Secondary peristalsis returns approximately 90% of the acid and food to the stomach. Once peristalsis ends, the LES closes again. The remaining acid in the esophagus is neutralized by successive swallows of saliva, which is alkaline in nature, and then cleared into the stomach.

Gastroesophageal reflux is both a normal physiologic phenomenon that occurs in the general population and a pathophysiologic phenomenon that can result in mild to severe symptoms. GERD can be described as any symptomatic clinical condition or change in tissue structure that results from the reflux of stomach or duodenal contents into the esophagus.

In patients with significant GERD, dysphagia is common and may be a sign of the formation of a stricture in the esophagus. Pulmonary manifestations such as asthma, coughing, or intermittent wheezing and vocal cord inflammation with hoarseness occur in some patients.

Complications of GERD include esophageal erosion, esophageal ulcer, and esophageal stricture; replacement of normal esophageal epithelium with abnormal (Barrett's) epithelium; and pulmonary aspiration.

The ability to target treatment of GERD to the esophagus is highly desirable.

c. Infections

Active agent compositions useful in treating infections of a foreign agent, such as viral infections, bacterial infections, prion infections, etc., can comprise: (1) at least one active agent, such as an antibiotic or antiviral agent; (2) at least one PEG-derivatized surface stabilizer; and (3) at least one antibody or a fragment thereof which specifically binds to the infectious agent. Such compositions, having high binding affinity for the infectious agent, can result in reduced active agent dosage and side effects as compared to conventional therapies.

An example of an infection that can be treated using the compositions of the invention is Helicobacter pylori infection. A composition according to the invention for treating H. pylori infection can comprise an antibody or fragment thereof which specifically binds to H. pylori.

H. pylori is a spiral shaped bacterium that lives in the stomach and duodenum (the section of intestine just below the stomach). It used to be thought that the stomach contained no bacteria and, was actually sterile, but H. pylori changed that.

Nearly all persons with duodenal ulcer are infected with H. pylori. Conversely, it is very unlikely that persons without H. pylori will ever develop duodenal ulcer. Gastric ulcer is usually caused by H. pylori, but about 30% of gastric ulcers in the United States occur in persons without H. pylori and can be related to aspirin and other non steroidal anti-inflammatory drugs (NSAIDs). Most gastric adenocarcinomas and lymphomas occur in persons with current or past infection with H. pylori.

Duodenal peptic ulcers occur in the first part of the intestine, one of two inches past the end of the stomach. Most duodenal ulcers occur in patients with H. pylori infection. If duodenal ulcers are treated with antacids or drugs such as cimetidine, ranitidine, and samotidine, they usually come back when the drugs are stopped. Acid reducing drugs are expensive and do not cure the duodenal ulcer problem. It has now been proven that by killing H. pylori, many patients with duodenal ulcer can be cured. After killing the H. pylori germ, most patients (80%) will be able to stop taking acid reducing drugs.

The most common cause of peptic ulcers is H. pylori infection of the stomach. It is expected that stomach ulcers will behave similar to duodenal ulcers so that after killing the H. pylori, they should not recur. Stomach ulcers are more complicated than duodenal ulcers, however, but the effectiveness of antibiotic treatment for stomach ulcers appears to be similar to that seen in duodenal ulcers (cure rate 70-90% if H. pylori is eradicated).

About 30% of stomach ulcers are not caused by H. pylori but are due to the corrosive effect of aspirin type medications, such as are taken for arthritis. These stomach ulcers may benefit from antibiotic treatment if H. pylori is also present.

Stomach cancers (gastric adenocarcinomas) are often associated with H. pylori (70-90%). In an extensive review of gastric cancer and H. pylori, the Eurogast Study Group determined that presence of H. pylori confers an approximately six fold risk of gastric cancer, accounting for about half of all gastric cancers. Supposedly, chronic gastritis leads to intestinal metaplasia (atrophic gastritis) which then undergoes malignant change. In the final stage H. pylori may no longer be detected on biopsy but immunologic studies may show evidence of past infection.

A composition according to the invention for treating stomach cancers, whether or not related to H. pylori infection, can comprise an antibody or fragment thereof which specifically binds to a cancer cell.

Mucosa associated lymphoid tissue (MALT) may undergo malignant change causing a low-grade lymphoma of the stomach. Retrospective biopsy studies show that 90% of such MALT lymphomas are associated with H. pylori. Early reports indicate about a 50% cure for localized MALT after cure of H. pylori. A composition according to the invention for treating MALT can comprise an antibody or fragment thereof which specifically binds to MALT tissue.

In patients with chronic dyspepsia who do not have ulcer disease, the role of H. pylori therapy has not been proven. In some patients an immediate response is seen but in others gradual improvement occurs over several months. There are several reports indicating that patients with chronic vomiting remit after H. pylori is eradicated.

There are several miscellaneous conditions that might be caused or worsened by H. pylori. Acne rosacea is a red skin rash on the face, it may respond to H. pylori therapy. Patients with H. pylori have increased permeability of the gastric mucosa and so are potentially exposed to unprocessed antigens from food. This might predispose to immune problems. H. pylori antibodies cross react with several tissues in the GUT so autoimmune states are possible with H. pylori. Skin rashes have occasionally disappeared when H. pylori was treated. Many patients experience improved well being and energy level when H. pylori is treated, so it is considered in treatment of Gulf Veterans Syndrome and Chronic Fatigue Syndrome. Many people with chronic halitosis respond to treatment for H. pylori. This may be because mouth bacteria and sinus and periodontal disease responds to the same antibiotics. It may be that H. pylori is the cause of the halitosis (bad digestion, achlorhydria etc.). See www.helico.com.

The ability to specifically target an antibiotic to H. pylori could dramatically increase the effectiveness of treatment.

d. Inflammation

Inflammation is a defense reaction caused by tissue damage or injury, characterized by redness, heat, swelling, and pain. The primary objective of inflammation is to localize and eradicate the irritant and repair the surrounding tissue. For the survival of the host, inflammation is a necessary and beneficial process. The inflammatory response involves three major stages: first, dilation of capillaries to increase blood flow; second, microvascular structural changes and escape of plasma proteins from the bloodstream; and third, leukocyte transmigration through endothelium and accumulation at the site of injury.

The leukocyte adhesion cascade is a sequence of adhesion and activation events that ends with extravasation of the leukocyte, whereby the cell exerts its effects on the inflamed site.

The roles of adhesion molecules in acute and chronic inflammation have been investigated using in vitro model systems and in vivo microcirculation studies. The ultimate goal of inflammation research is to develop methods to control inflammation by modulating or blocking leukocyte adhesion to the endothelium. These ideas developed by basic research contribute to contemporary research projects developing anti-inflammatory drugs. Anti-inflammatory agents function as blockers, suppressors, or modulators of the inflammatory response.

The ability to target anti-inflammatory drugs to a desired site could significantly increase their effectiveness, as well as allow for a decreased dosage. Such a decreased dosage could produce fewer side effects, such as the stomach irritation associated with NSAIDs.

e. Epithelial Cells

The target agent can be any biological target, such as, for example, an epithelial cell. Epithelial cells can be, for example, from epithelial tissues such as anterior epithelium of cornea, Barrett epithelium, ciliated epithelium, columnar epithelium, crevicular epithelium, cuboidal epithelium, epithelium of semicircular duct, germinal epithelium, gingival epithelium, glandular epithelium, stratified epithelium, epithelium of lens, mesenchymal epithelium, muscle epithelium, olfactory epithelium, simple squamous epithelium, pigment epithelium, pseudostratified epithelium, respiratory epithelium, seminiferous epithelium, simple epithelium, stratified ciliated columnar epithelium, stratified squamous epithelium, surface epithelium, and transitional epithelium.

In sum, in one aspect of the invention the compositions are useful in treating any epithelial cell surface.

B. Increased Bioavailability

In a preferred embodiment of the invention, the active agent compositions exhibit increased bioavailability, at the same dose of the same active agent, and require smaller doses as compared to prior conventional active agent formulations. This is because the compositions of the invention enable targeting of the active agent, which results in substantial dissolution of the active agent at the target site.

The active agent compositions of the invention enable delivery of concentrated active agent in a small dosage volume because of the solid state nature of the active agent in a dispersion or solid dose. In contrast, microparticulate active agents in solution require a much larger dosage volume for delivery of the same quantity of active agent. A smaller solid dosage size or volume is particularly significant for patient populations such as the elderly, juvenile, and infant.

A solid active agent particle comprises many molecules of active agent, whereas solubilized active agent is delivered to the target site molecule by molecule. The multitude of molecules delivered at one time with a solid particle results in faster onset of therapeutic activity; i.e. the active agent compositions of the invention provide more active agent molecules per delivery unit as compared to conventional microparticulate solution active agent formulations.

Enhanced bioavailability enables the use of lower doses, which also results in decreased toxicity associated with the active agent. In this regard, lower doses of the active agent when formulated into the compositions of the invention can achieve the same or better therapeutic effects as larger doses of conventional forms of the same active agent. Such lower doses can be realized due to the greater bioavailability of compositions of the invention as compared to conventional active agent formulations. Most active agents can have adverse side effects. Therefore, the ability to administer lower doses of an active agent translates into fewer adverse side effects.

C. Improved Pharmacokinetic Profiles

The inventive active agent compositions also preferably exhibit a desirable pharmacokinetic profile when administered to mammalian subjects. The desirable pharmacokinetic profile preferably includes, but is not limited to: (1) that the Tₘₐₓ of the active agent, when assayed in the plasma of a mammalian subject following administration, is preferably less than the Tₘₐₓ for a conventional, non-nanoparticulate form of the same active agent, administered at the same dosage; (2) that the Cₘₐₓ of the active agent, when assayed in the plasma of a mammalian subject following administration, is preferably greater than the Cₘₐₓ for a conventional, non-nanoparticulate form of the same active agent, administered at the same dosage; and/or (3) that the AUC of the active agent, when assayed in the plasma of a mammalian subject following administration, is preferably greater than the AUC for a conventional, non-nanoparticulate form of the same active agent administered at the same dosage.

The desirable pharmacokinetic profile, as used herein, is the pharmacokinetic profile measured after an initial dose of the active agent. The dose can be formulated in any way as described below and as known to those skilled in the art.

A preferred active agent composition exhibits, in comparative pharmacokinetic testing with a non-nanoparticulate formulation of the same active agent administered at the same dosage, a Tₘₐₓ not greater than about 100%, not greater than about 90%, not greater than about 80%, not greater than about 70%, not greater than about 60%, not greater than about 50%, not greater than about 40%, not greater than about 30%, not greater than about 25%, not greater than about 20%, not greater than about 15%, or not greater than about 10% of the Tₘₐₓ, exhibited by the non-nanoparticulate formulation of the same active agent.

A preferred active agent composition exhibits, in comparative pharmacokinetic testing with a non-nanoparticulate formulation of the same active agent administered at the same dosage, a Cₘₐₓ that is at least about 10%, at least about 20%, at least about 30%, at least about 400%, at least about 50%, at least about 60%, at least about 70%, at least about 80%, at least about 90%, or at least about 100% greater than the Cₘₐₓ exhibited by the non-nanoparticulate formulation of the same active agent.

A preferred active agent composition exhibits, in comparative pharmacokinetic testing with a non-nanoparticulate formulation of the same active agent administered at the same dosage, an AUC that is at least about 10%, at least about 20%, at least about 30%, at least about 40%, at least about 50%, at least about 60%, at least about 70%, at least about 80%, at least about 90%, or at least about 100% greater than the AUC exhibited by the non-nanoparticulate formulation of the same active agent.

According to the invention, any formulation that provides the desired pharmacokinetic profile is suitable for administration. Exemplary types of formulations that give such profiles are liquid dispersions, gels, aerosols, ointments, creams and solid dose forms.

D. The Pharmacokinetic Profiles of the Active Agent Compositions of the Invention are not Affected by the Fed or Fasted State of the Subject Ingesting the Compositions

Certain drugs have been shown to have significantly lower plasma levels when administered under fasting conditions as compared to administration immediately after a standard test meal. This significant difference is undesirable.

Nanoparticulate active agent compositions of the invention preferably alleviate this problem. That is, the compositions of the invention preferably reduce the differences in, or more preferably do not produce significantly different, absorption levels when administered under fed as compared to fasting conditions.

Thus, the invention encompasses an active agent composition having a pharmacokinetic profile that is not. substantially affected by the fed or fasted state of a subject ingesting the composition. This means that there is no substantial difference in the quantity of active agent absorbed or the rate of active agent absorption when the active agent compositions of the invention are administered in the fed versus the fasted state.

The invention also encompasses an active agent composition for which administration to a subject in a fasted state is bioequivalent to administration to a subject in a fed state. “Bioequivalency” is preferably established by a 90% Confidence Interval (CI) of between 0.80 and 1.25 for both Cₘₐₓ and AUC under U.S. Food and Drug Administration regulatory guidelines, or a 90% CI for AUC of between 0.80 to 1.25 and a 90% CI for Cₘₐₓ of between 0.70 to 1.43 under the European EMEA regulatory guidelines (Tₘₐₓ is not relevant for bioequivalency determinations under USFDA and EMEA regulatory guidelines).

The difference in absorption (AUC), Cₘₐₓ, and Tₘₐₓ of the inventive active agent compositions, when administered in a fed versus a fasted state, preferably is less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, or less than about 3%.

Benefits of a dosage form that substantially eliminates the effect of food include an increase in convenience, which increases patient compliance, as a patient does not need to ensure that they are taking a dose either with or without food. This is significant, as poor patient compliance can defeat the purpose of administering an active agent.

E. Dissolution Profiles of the Active Agent Compositions of the Invention

In a preferred embodiment of the invention, the active agent compositions of the invention, when formulated into a solid dose, have unexpectedly dramatic dissolution profiles. Rapid dissolution of an administered active agent is preferable, as faster dissolution generally leads to faster onset of action and greater bioavailability.

The active agent compositions ofthe invention, when formulated into a solid dose, preferably have a dissolution profile in which within about 5 minutes at least about 20% of the composition is dissolved. In other embodiments of the invention, preferably at least about 30% or about 40% of the active agent composition is dissolved within about 5 minutes. In yet other embodiments of the invention, preferably at least about 40%, about 50%, about 60%, about 70%, or about 80% of the active agent composition is dissolved within about 10 minutes. Finally, in another embodiment of the invention, preferably at least about 70%, about 80%, about 90%, or about 100% of the active agent composition is dissolved within about 20 minutes.

Dissolution is preferably measured in a media which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

F. Redispersibility Profiles of the Active Agent Compositions of the Invention

Preferably, the compositions of the invention redisperse such that the effective average particle size of the redispersed active agent particles is less than about 2 microns. This is significant, as if upon administration the active agent compositions of the invention did not redisperse to a particle size which is substantially similar to that prior to incorporation into the dosage form, then the dosage form may lose the benefits afforded by formulating the active agent into a small particle composition.

This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the active agent compositions of the invention exhibit dramatic redispersion of the active agent particles upon administration to a mammal, such as a human or animal, as demonstrated by reconstitution/redispersion in a biorelevant aqueous media such that the effective average particle size of the redispersed active agent particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M, while fasted state intestinal fluid has an ionic strength of about 0.14 M. See e.g., Lindahl et.al., “Characterization of Fluids from the Stomach and Proximal Jejunum in Men and Women,” Pharm. Res., 14 (4): 497-502 (1997).

It is believed that the pH and ionic strength ofthe test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed active agent particles of the invention (redispersed in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example section of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

G. Bioadhesive Active Agent Compositions

Active agent compositions of the invention can exhibit bioadhesive properties. Such compositions comprise one or more cationic surface stabilizers, which are described in more detail below. The cationic surface stabilizer can be PEG-derivatized, or it can be a second surface stabilizer used in conjunction with a PEG-derivatized surface stabilizer.

The term bioadhesion refers to any attractive interaction between two biological surfaces or between a biological and a synthetic surface. In the case of bioadhesive active agent compositions, the term bioadhesion describes the adhesion between active agent compositions and a biological substrate (e.g., gastrointestinal mucin, lung tissue, nasal mucosa, etc.). See, e.g., U.S. Pat. No. 6,428,814 for “Bioadhesive Nanoparticulate Compositions Having Cationic Surface Stabilizers,” which is specifically incorporated by reference. Bioadhesive formulations of active agents according to the invention exhibit exceptional bioadhesion to biological substrates.

Bioadhesive active agent compositions are useful in any situation where it is desirable to apply the compositions to a biological surface. Bioadhesive active agent compositions coat the targeted surface in a continuous and uniform film, which is invisible to the naked human eye.

Bioadhesion provides a prolonged exposure to between the target cells of the invention and the active agent, thereby increasing absorption and bioavailability of the administered dosage. Bioadhesive compositions of the invention are particularly beneficial, as the bioadhesive aspect coupled with the targeting aspect of the compositions can provide for dramatic therapeutic effectiveness.

H. Low Viscosity Liquid Active Agent Compositions

A liquid dosage form of a conventional microcrystalline or non-nanoparticulate, or solubilized, active agent composition would be expected to be a relatively large volume, highly viscous substance which would not be well accepted by patient populations. This is significant, as liquid dosage forms can be particularly useful for patient populations such as the elderly, pediatric, and infant.

Liquid dosage forms of the active agent compositions of the invention provide significant advantages over a liquid dosage form of a conventional microcrystalline or solubilized active agent composition. The low viscosity and silky texture of liquid dosage forms of the active agent compositions of the invention result in advantages in both preparation and use. These advantages include, for example: (1) better subject compliance due to the perception of a lighter formulation which is easier to ingest; (2) ease of dispensing as compared to a highly viscous formulation; (3) potential for formulating a higher concentration of the active agent resulting in a smaller dosage volume and thus less volume for the subject to consume; and (4) easier overall formulation concerns.

The viscosities of liquid dosage forms of the active agent compositions according to the invention are preferably less than about 1/200, less than about 1/175, less than about 1/150, less than about 1/125, less than about 1/100, less than about 1/75, less than about 1/50, or less than about 1/25 of a topical liquid dosage form of a non-nanoparticulate composition of the same active agent, at about the same concentration per ml of active agent.

Typically the viscosity of liquid active agent dosage forms of the invention, at a shear rate of 0.1 (1/s) and measured at 20° C., is from about 2000 mPa s to about 1 mPa s, from about 1900 mPa.s to about 1 mPa.s, from about 1800 mPa.s to about 1 mPa.s, from about 1700 mPa.s to about 1 mPa.s, from about 1600 mPa.s to about 1 mPa.s, from about 1500 mPa.s to about 1 mPa.s, from about 1400 mPa.s to about 1 mPa.s, from about 1300 mPa.s to about 1 mPa.s, from about 1200 mPa.s to about 1 mPa.s, from about 1100 mPa.s to about 1 mPa.s, from about 1000 mPa.s to about 1 mPa.s, from about 900 mPa.s to about 1 mPa.s, from about 800 mPa.s to about 1 mPa.s, from about 700 mPa.s to about 1 mPa.s, from about 600 mPa.s to about 1 mPa.s, from about 500 mPa.s to about 1 mPa.s, from about 400 mPa.s to about 1 mPa.s, from about 300 mPa.s to about 1 mPa.s, from about 200 mPa.s to about 1 mPa.s, from about 175 mPa.s to about 1 mPa.s, from about 150 mPa.s to about 1 mPa.s, from about 125 mPa.s to about 1 mPa.s, from about 100 mPa.s to about 1 mPa.s, from about 75 mPa.s to about 1 mPa.s, from about 50 mPa.s to about 1 mPa.s, from about 25 mPa.s to about 1 mPa.s, from about 15 mPa.s to about 1 mPa.s, from about 10 mPa.s to about 1 mPa.s, or from about 5 mPa.s to about 1 mPa.s.

Viscosity is concentration and temperature dependent. Typically, a higher concentration results in a higher viscosity, while a higher temperature results in a lower viscosity. Viscosity as defined above refers to measurements taken at about 20° C. (The viscosity of water at 20° C. is 1 mPa s.) The invention encompasses equivalent viscosities measured at different temperatures.

The liquid formulations of this invention can be formulated for dosages in any volume but are preferably equivalent or smaller volumes than a liquid dosage form of a non-nanoparticulate or solubilized composition of the same active agent.

I. Combination Pharmacokinetic Profile Compositions

In one embodiment of the invention, a first active agent composition providing the pharmacokinetic profile described above is co-administered with at least one other active agent composition that generates a different pharmacokinetic profile, specifically one exhibiting slower absorption into the bloodstream, and therefore a longer Tₘₐₓ and typically a lower Cₘₐₓ. The second composition can comprise the same or a different active agent.

For example, the second active agent formulation can have a conventional particle size, which produces a longer Tₘₐₓ, and typically a lower Cₘₐₓ. Alternatively, a second, third or fourth active agent composition can differ from the first, and from each other, in for example: (1) the effective average particle sizes of each composition; (2) the concentration of active agent of each composition;, or (3) the identity of the active agent for each composition. The difference particle sizes produce different Tₘₐₓ values. The combination of fast pain relief provided by the first formulation and longer-lasting pain relief provided by the second (or third, fourth, etc.) formulation can reduce the dose frequency required.

Preferably where co-administration of a “fast-acting” formulation and a “longer-lasting” formulation is desired, the two formulations are combined within a single composition, for example a dual-release composition.

II. Compositions

The invention provides compositions comprising: (1) one or more antibodies or a fragment thereof; (2) one or more active agents; and (3) one or more PEG-derivatized surface stabilizers.

A. Antibodies or Fragments Thereof

The antibody or fragment thereof can be attached to the PEG-derivatized surface stabilizer, either directly or indirectly, by procedures well known in the art.

1. Antibodies or Fragments Thereof Useful in the Invention

Any antibody or fragment thereof having the ability to specifically react with a target site (e.g., analyte, epitope, or antigen) is useful in the present invention. The antibody or fragment thereof can be from any of the five different classes of antibodies or immunoglobulins, e.g., IgD, IgA, IgM, IgE, and IgG. In addition, the antibody or fragment thereof utilized in the present invention can be from any of the four different subclasses of IgG or from either of the two different sublcasses of IgA.

The F or Fab region of an antibody is the portion of the immunoglobulin molecule which contains the binding site for antigens. The exact sequence of amino acids in the area varies widely from molecule to molecule to accommodate a wide variety of antigens which the body may encounter. There are two such regions on each molecule (individually called Fab fragments or F-ab fragments). The molecule is shaped like the letter Y and the F(ab)2 fragments are located on the upper halves of the two fork parts. (The rest of the molecule, the stem and the lower parts of the forks, are the Fc fragment). An antibody fragment according to the invention can be, for example, one or more Fab regions of an antibody, or a part of a Fab region having binding ability, such as a VL or VH region. F(ab′)₂, Fab, Fab′, and Fv are exemplary antigen binding fragments that can be generated from the variable region of immunoglobulin.

Antibody fragments offer several advantages over intact antibody. For example, antibody fragments offer reduced non-specificity resulting from Fc interactions (many cells have receptors for binding to the Fc portion of antibodies). In addition, antibody fragments generally provide higher sensitivity in antigen detection for solid phase applications as a result of reduced steric hindrance from large protein epitopes. In addition, antibody fragments are the best choice for antigen-antibody binding studies in the absence of Fc associated effector functions (for example, complement fixation and cell membrante receptor interaction). Moreover, antibody fragments possess improved biodistribution properties, such as tumor tissue penetration. Antibody fragments offer lower immunogenicity than intact antibody, such fragments can more easily cross capillaries and diffuse to tissue surfaces, and antibody fragments not bound to conjugate will be cleared more rapidly than intact immunoglobulin; therefore, more of the fragment-therapeutic agent will reach the target area. Removal of the Fc region makes the antibody fragment less susceptible to catabolism by phagocytic cells that have Fc receptors. Finally, antibody fragments are useful in immunohistochemical studies because the fragments penetrate tissue better than intact immunoglobulin, avoiding non-specific binding due to Fc receptors. See Pierce Life Science and Analytical Research Products (Pierce Chemical Co. 1994).

As used herein, the term “antibody” includes reference to antigen binding forms of antibodies (e.g., Fab, F(ab)₂). The term “antibody” frequently refers to a polypeptide substantially encoded by an immunoglobulin gene or immunoglobulin genes, or fragments thereof which specifically bind and recognize a target site (e.g., analyte, epitope, or antigen). However, while various antibody fragments can be defined in terms of the digestion of an intact antibody, one of skill will appreciate that such fragments may be synthesized de novo either chemically or by utilizing recombinant DNA methodology. Thus, the term antibody as used herein also includes antibody fragments such as single chain Fv, chimeric antibodies (i.e., comprising constant and variable regions from different species), humanized antibodies (i.e., comprising a complementary determining region (CDR) from a non-human source), and heteroconjugate antibodies (e.g., bispecific antibodies).

The terms “target site”, “analyte”, and “antigen” include reference to a substance to which an antibody can be generated and/or to which the antibody is specifically immunoreactive. The specific immunoreactive sites within the antigen are known as epitopes or antigenic determinants. These epitopes can be a linear array of monomers in a polymeric composition—such as amino acids in a protein—or consist of or comprise a more complex secondary or tertiary structure. Those of skill will recognize that all immunogens (i.e., substances capable of eliciting an immune response) are antigens; however some antigens, such as haptens, are not immunogens but may be made immunogenic by coupling to a carrier molecule. An antibody immunologically reactive with a particular antigen can be generated in vivo or by recombinant methods such as selection of libraries of recombinant antibodies in phage or similar vectors. See e.g., Huse et al., Science, 246:1275-1281 (1989); Ward et al., Nature, 341:544-546 (1989); and Vaughan et al., Nature Biotech., 14: 309-314 (1996).

“Immunologically reactive conditions” or “immunoreactive conditions” mean conditions which allow an antibody, reactive to a particular epitope, to bind to that epitope to a detectably greater degree (e.g., at least 2-fold over background) than the antibody binds to substantially any other epitopes in a reaction mixture comprising the particular epitope. Immunologically reactive conditions are dependent upon the format of the antibody binding reaction and typically are those utilized in immunoassay protocols. See Harlow and Lane, Antibodies, A Laboratory Manual, Cold Spring Harbor Publications, New York (1988), for a description of exemplary immunoassay formats and conditions.

The term “specifically reactive” includes reference to a binding reaction between an antibody and a target site having an epitope recognized by the antigen binding site of the antibody.

2. Exemplary Commercial Sources of Antibodies or Fragments Thereof

There are many commercial sources of antibodies and antibody fragments. See e.g., http://www.antibodyresource.com/onlinecomp.html, which provides a list of 247 on-line companies which sell antibodies, such as Abcam Ltd. (www.abcam.com), Fusion Antibodies Ltd. (Belfast, N. Ireland; www/fusionantibodies.com); Abgent (San Diego, Calif.; www.abgent.com); Abkem Iberia (Vigo, Spain; http://www.abkemiberia.com/); Academy Bio-Medical Co., Inc. (Houston, Tex.; www.academybiomed.com); and Accurate Chemical and Scientific Corp. (Westbury, N.Y.; www.accuratechemical.com).

There are also many commercial companies which supply custom monoclonal and polyclonal antibodies. At http://www.antibodyresource.com/customantibody.html, 125 such suppliers are listed.

3. Methods of Making Antibodies or Fragments Thereof Known in the Art

Many methods of making antibodies are known. See e.g., J. Janin, Nature, 277:491-492 (1979); Wolfenden et al., Biochemistry, 20:849-855 (1981); Kyte and Doolite, J. Mol. Biol., 157:105-132 (1982); and Rose et al., Science, 229:834-838 (1985). The following discussion is presented as a general overview of the techniques available; however, one of skill will recognize that many variations upon the following methods are known.

A number of immunogens can be used to produce antibodies specifically reactive with a target site of the present invention. For example, an isolated recombinant, synthetic, or native polynucleotide,can be used as an antigen for the production of monoclonal or polyclonal antibodies. Polypeptides are optionally denatured, and optionally reduced, prior to formation of antibodies for screening expression libraries or other assays in which a putative target site of the present invention is expressed or denatured in a non-native secondary, tertiary, or quarternary structure.

In addition, antibodies can be raised to a desired target site, including individual, allelic, strain, or species variants of such target sites, both in its naturally occurring (full-length) form and in a recombinant form. Antibodies can be raised to a target site in either its native configuration or in a non-native configuration.

The target site (e.g., analyte, antigen, protein, etc.) of the present invention is then injected into an animal capable of producing antibodies. Either monoclonal or polyclonal antibodies can be generated for subsequent use in the compositions of the invention. Preferably, monoclonal antibodies are utilized in the compositions of the invention.

Methods of producing polyclonal antibodies are known to those of skill in the art. In brief, an antigen, analyte, or target site, such as a purified protein, a protein coupled to an appropriate carrier (e.g., GST, keyhole limpet hemanocyanin, etc.), or a protein incorporated into an immunization vector such as a recombinant vaccinia virus (see U.S. Pat. No. 4,722,848), is mixed with an adjuvant and animals are immunized with the mixture. The animal's immune response to the immunogen preparation is monitored by taking test bleeds and determining the titer of reactivity to the protein of interest. When appropriately high titers of antibody to the immunogen are obtained, blood is collected from the animal and antisera are prepared. Further fractionation of the antisera to enrich for antibodies reactive to the protein is performed where desired (See e.g., Coligan, Current Protocols in Immunology, Wiley/Greene, NY (1991); and Harlow and Lane, Antibodies: A Laboratory Manual, Cold Spring Harbor Press, NY (1989)).

Monoclonal antibodies are prepared from hybrid cells secreting the desired antibody. Monoclonal antibodies are screened for binding to a protein from which the antigen was derived. Specific monoclonal and polyclonal antibodies will usually have an antibody binding site with an affinity constant for its cognate monovalent antigen at least between 10⁶−10⁷, usually at least 10⁸, preferably at least 10⁹, more preferably at least 10¹⁰, and most preferably at least 10¹¹ liters/mole.

A variety of immunoassay formats may be used to select antibodies (monoclonal or polyclonal) specifically reactive with a particular target site. For example, solid-phase ELISA immunoassays are routinely used to select monoclonal antibodies specifically immunoreactive with a protein. See Harlow and Lane, Antibodies, A Laboratory Manual, Cold Spring Harbor Publications, New York (1988), for a description of immunoassay formats and conditions that can be used to determine selective reactivity. Other exemplary and well known in the art immunoassay formats include competitive immunoassays, radioimmunoassays, Western blots, indirect immunofluorescent assays, and the like.

In some instances, it is desirable to prepare monoclonal antibodies from various mammalian hosts, such as mice, rodents, primates, humans, etc. A description of techniques for preparing such monoclonal antibodies is found in, e.g., Basic and Clinical Immunology, 9th ed., Stites et al., Eds. (Appleton & Lange Publications, San Mateo, Calif., 1998), and references cited therein; Harlow and Lane, Antibodies, A Laboratory Manual, Cold Spring Harbor Publications, New York (1988); Goding, Monoclonal Antibodies: Principles and Practice, 2nd ed. (Academic Press, New York, N.Y. 1986); and Kohler and Milstein, Nature, 256:495-497 (1975). Summarized briefly, this method proceeds by injecting an animal with an antigen (i.e., target site or analyte). The animal is then sacrificed and cells are taken from its spleen, which are fused with myeloma cells. The result is a hybrid cell or “hybridoma” that is capable of reproducing in vitro. The population of hybridomas is then screened to isolate individual clones, each of which secrete a single antibody species to the target site or antigen. In this manner, the individual antibody species obtained are the products of immortalized and cloned single B cells from the immune animal generated in response to a specific site recognized on the antigenic substance.

Other suitable techniques involve selection of libraries of recombinant antibodies in phage or similar vectors (see e.g., Huse et al., Science, 246:1275-1281 (1989); Ward et al., Nature, 341:544-546 (1989); and Vaughan et al., Nature Biotechnology, 14:309-314 (1996)). Alternatively, high avidity human monoclonal antibodies can be obtained from transgenic mice comprising fragments of the unrearranged human heavy and light chain Ig loci (i.e., minilocus transgenic mice). See Fishwild et al., Nature Biotech., 14: 845-851 (1996). Also, recombinant immunoglobulins may be produced. See Cabilly, U.S. Pat. No. 4,816,567; and Queen et al., Proc. Nat'l Acad. Sci., 86:10029-10033 (1989).

Finally, a fragment of an antibody protein which includes the antigen-binding portions but not the Fc section can be produced by treating whole antibodies with proteases that will specifically cleave off the Fc section.

B. Active Agents

The invention can be practiced with a wide variety of active agents. For example, the compositions of the invention can comprise at least one active, therapeutic, or diagnostic agent, collectively referred to as a “drug.” A therapeutic agent can be a pharmaceutical agent, including biologics such as proteins, peptides, and nucleotides, or a diagnostic agent, such as a contrast agent, including x-ray contrast agents.

The active agent exists either as a discrete, crystalline phase, a semi-crystalline phase, an amorphous phase, a semi-amorphous phase, or a combination thereof.

Two or more active agents can be used in combination. In addition, the compositions of the invention can be co-administered, sequentially administered, or co-formulated with a second (or third; fourth, etc.) active agent which is conventional, meaning that the active agent is not associated with a PEG-derivatized surface stabilizer and antibody or fragment thereof.

The active agent is preferably poorly soluble and dispersible in at least one liquid dispersion media. By “poorly soluble” it is meant that the active agent has a solubility in a liquid dispersion media of less than about 30 mg/mL, less than about 20 mg/mL, less than about 10 mg/mL; or less than about 1 mg/mL. Useful liquid dispersion media include, but are not limited to, water, aqueous salt solutions, safflower oil, and solvents such as ethanol, t-butanol, hexane, and glycol. A preferred liquid dispersion media is water.

1. Active Agents Generally

The active agent can be selected from a variety of known classes of drugs, including, for example, nutraceuticals, COX-2 inhibitors, retinoids, anticancer agents, NSAIDS, proteins, peptides, nucleotides, anti-obesity drugs, nutraceuticals, dietary supplements, carotenoids, corticosteroids, elastase inhibitors, anti-fungals, oncology therapies, anti-emetics, analgesics,. cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Examples of representative active agents useful in this invention include, but are not limited to, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, and acetylsalicylate.

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. A nutraceutical or dietary supplement, also known as phytochemicals or functional foods, is generally any one of a class of dietary supplements, vitamins, minerals, herbs, or healing foods that have medical or pharmaceutical effects on the body. Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

Active agents to be administered in an aerosol formulation are preferably selected from the group consisting of proteins, peptide, bronchodilators, corticosteroids, elastase inhibitors, analgesics, anti-fungals, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, flngal infection therapies, respiratory illness therapies associated with acquired immune deficiency syndrome, an oncology drug, an anti-emetic, an analgesic, and a cardiovascular agent.

A description of these classes of active agents and a listing of species within each class can be found in Martindale, The Extra Pharmacopoeia, Twenty-ninth Edition (The Pharmaceutical Press, London, 1989), specifically incorporated by reference. The active agents are commercially available and/or can be prepared by techniques known in the art.

2. Anticancer Active Agents

Useful anticancer agents are preferably selected from alkylating agents, antimetabolites, natural products, hormones and antagonists, and miscellaneous agents, such as radiosensitizers.

Examples of alkylating agents include: (1) alkylating agents having the bis-(2-chloroethyl)-amine group such as, for example, chlormethine, chlorambucile, melphalan, uramustine, mannomustine, extramustinephoshate, mechlore-thaminoxide, cyclophosphamide, ifosfamide, and trifosfamide; (2) alkylating agents having a substituted aziridine group such as, for example, tretamine, thiotepa, triaziquone, and mitomycine; (3) alkylating agents of the alkyl sulfonate type, such as, for example, busulfan, piposulfan, and piposulfam; (4) alkylating N-alkyl-N-nitrosourea derivatives, such as, for example, carmustine, lomustine, semustine, or streptozotocine; and (5) alkylating agents of the mitobronitole, dacarbazine and procarbazine type.

Examples of antimetabolites include: (1) folic acid analogs, such as, for example, methotrexate; (2) pyrimidine analogs such as, for example, fluorouracil, floxuridine, tegafur, cytarabine, idoxuridine, and flucytosine; and (3) purine derivatives such as, for example, mercaptopurine, thioguanine, azathioprine, tiamiprine, vidarabine, pentostatin, and puromycine.

Examples of natural products include: (1) vinca alkaloids, such as, for example, vinblastine and vincristine; (2) epipodophylotoxins, such as, for example, etoposide and teniposide; (3) antibiotics, such as, for example, adriamycine, daunomycine, doctinomycin, daunorubicin, doxorubicin, mithramycin, bleomycin, and mitomycin; (4) enzymes, such as, for example, L-asparaginase; (5) biological response modifiers, such as, for example, alpha-interferon; (6) camptothecin; (7) taxol; and (8) retinoids, such as retinoic acid.

Examples of hormones and antagonists include: (1) adrenocorticosteroids, such as, for example, prednisone; (2) progestins, such as, for example, hydroxyprogesterone caproate, medroxyprogesterone acetate, and megestrol acetate; (3) estrogens, such as, for example, diethylstilbestrol and ethinyl estradiol; (4) antiestrogens, such as, for example, tamoxifen; (5) androgens, such as, for example, testosterone propionate and fluoxymesterone; (6) antiandrogens, such as, for example, flutamide; and (7) gonadotropin-releasing hormone analogs, such as, for example, leuprolide.

Examples of miscellaneous agents include: (1) radiosensitizers, such as, for example, 1,2,4-benzotriazin-3-amine 1,4-dioxide (SR 4889) and 1,2,4-benzotriazine-7-amine 1,4-dioxide (WIN 59075); (2) platinum coordination complexes such as cisplatin and carboplatin; (3) anthracenediones, such as, for example, mitoxantrone; (4) substituted ureas, such as, for example, hydroxyurea; and (5) adrenocortical suppressants, such as, for example, mitotane and aminoglutethimide.

In addition, the anticancer agent can be an immunosuppressive drug, such as, for example, cyclosporine, azathioprine, sulfasalazine, methoxsalen, and thalidomide.

The anticancer agent can also be a COX-2 inhibitor.

3. Analgesics

An analgesic can be, for example, an NSAID or a COX-2 inhibitor.

Exemplary NSAIDS that can be formulated in compositions of the invention include, but are not limited to, suitable nonacidic and acidic compounds. Suitable nonacidic compounds include, for example, nabumetone, tiaramide, proquazone, bufexamac, flumizole, epirazole, tinoridine, timegadine, and dapsone. Suitable acidic compounds include, for example, carboxylic acids and enolic acids. Suitable carboxylic acid NSAIDs include, for example: (1) salicylic acids and esters thereof, such as aspirin, diflunisal, benorylate, and fosfosal; (2) acetic acids, such as phenylacetic acids, including diclofenac, aldlofenac, and fenclofenac; (3) carbo- and heterocyclic acetic acids such as etodolac, indomethacin, sulindac, tolmetin, fentiazac, and tilomisole; (4) propionic acids, such as carprofen, fenbufen, flurbiprofen, ketoprofen, oxaprozin, suprofen, tiaprofenic acid, ibuprofen, naproxen, fenoprofen, indoprofen, and pirprofen; and (5) fenamic acids, such as flufenamic, mefenamic, meclofenamic, and niflumic. Suitable enolic acid NSAIDs include, for example: (1) pyrazolones such as oxyphenbutazone, phenylbutazone, apazone, and feprazone; and (2) oxicams such as piroxicam, sudoxicam, isoxicam, and tenoxicam.

Exemplary COX-2 inhibitors that can be formulated in combination with the nanoparticulate nimesulide composition of the invention include, but are not limited to, celecoxib (SC-58635, CELEBREX®, Pharmacia/Searle & Co.), rofecoxib (MK-966, L-748731, VIOXX®, Merck & Co.), meloxicam (MOBIC®, co-marketed by Abbott Laboratories, Chicago, Ill., and Boehringer Ingelheim Pharmaceuticals), valdecoxib (BEXTRA®, G.D. Searle & Co.), parecoxib (G.D. Searle & Co.), etoricoxib (MK-663; Merck), SC-236 (chemical name of 4-[5-(4-chlorophenyl)-3-(trifluoromethyl)-1H-pyrazol-1-yl)] benzenesulfonamide; G.D. Searle & Co., Skokie, Ill.); NS-398 (N-(2-cyclohexyloxy-4-nitrophenyl)methane sulfonamide; Taisho Pharmaceutical Co., Ltd., Japan); SC-58125 (methyl sulfone spiro(2.4)hept-5-ene I; Pharmacia/Searle & Co.); SC-57666 (Pharmacia/Searle & Co.); SC-558 (Pharmacia/Searle & Co.); SC-560 (Pharmacia/Searle & Co.); etodolac (Lodine®, Wyeth-Ayerst Laboratories, Inc.); DFU (5,5-dimethyl-3-(3-fluorophenyl)-4-(4-methylsulfonyl)phenyl 2(5H)-furanone); monteleukast (MK-476), L-745337 ((5-methanesulphonamide-6-(2,4-difluorothio-phenyl)-1-indanone), L-761066, L-761000, L-748780 (all Merck & Co.); DUP-697 (5-Bromo-2-(4-fluorophenyl)-3-(4-(methylsulfonyl)phenyl; DuPont Merck Pharmaceutical Co.); PGV 20229 (1-(7-tert.-butyl-2,3-dihydro-3,3-dimethylbenzo(b)furan-5-yl)-4-cyclopropylbutan-1-one; Procter & Gamble Pharmaceuticals); iguratimod (T-614; 3-formylamino-7-methylsulfonylamino-6-phenoxy-4H-1-benzopyran-4-one; Toyama Corp., Japan); BF 389 (Biofor, USA); CL 1004 (PD 136095), PD 136005, PD 142893, PD 138387, and PD 145065 (all Parke-Davis/Warner-Lambert Co.); flurbiprofen (ANSAID®; Pharmacia & Upjohn); nabumetone (FELAFEN®; SmithKline Beecham, plc); flosulide (CGP 28238; Novartis/Ciba Geigy); piroxicam (FELDANE®; Pfizer); diclofenac (VOLTAREN® and CATAFLAM®, Novartis); lumiracoxib (COX-189; Novartis); D 1367 (Celltech Chiroscience, plc); R 807 (3 benzoyldifluoromethane sulfonanilide, diflumidone); JTE-522 (Japan Tobacco, Japan); FK-3311 (4′-Acetyl-2′-(2,4-difluorophenoxy)methanesulfonanilide), FK 867, FR 140423, and FR 115068 (all Fujisawa, Japan); GR 253035 (Glaxo Wellcome); RWJ 63556 (Johnson & Johnson); RWJ 20485 (Johnson & Johnson); ZK 38997 (Schering); S 2474 ((E)-(5)-(3,5-di-tert-butyl-4-hydroxybenzylidene)-2-ethyl-1,2-isothiazolidine-1,1-dioxide indomethacin; Shionogi & Co., Ltd., Japan); zomepirac analogs, such as RS 57067 and RS 104897 (Hoffmann La Roche); RS 104894 (Hoffmann La Roche); SC 41930 (Monsanto); pranlukast (SB 205312, Ono-1078, ONON®, ULTAIR®; SmithKline Beecham); SB 209670 (SmithKline Beecham); and APHS (heptinylsulfide).

4. Active Agents Useful in Dermal Applications

The active agents according to the present invention include, but are not limited to, active agents which can be used in dermal applications, e.g., sunscreens, cosmetics, topical application of pharmaceuticals to the dermis (acne medication, anti-wrinkle drugs, such as alpha-hydroxy formulations), moisturizers, deodorant, etc.

C. Surface Stabilizers

1. Primary Surface Stabilizer

The compositions of the invention comprise at least one PEG-derivatized surface stabilizer. By “PEG-derivatized,” it is meant that the surface stabilizer is modified by covalent attachment of at least one pendant PEG group.

The PEG-derivatized surface stabilizers of the invention are preferably adsorbed on, or associated with, the surface of the active agent particles. The PEG-derivatized surface stabilizers especially useful herein preferably do not chemically react with the active agent particles or itself. Preferably, individual molecules of the PEG-derivatized surface stabilizer are essentially free of intermolecular cross-linkages.

The PEG-derivatized surface stabilizer is adsorbed on or associated with the surface of the active agent in an amount sufficient to maintain the active agent particles at an effective average particle size of less than about 2 microns.

Two or more PEG-derivatized surface stabilizers can be employed in the compositions and methods of the invention.

Preferably, the PEG-derivatized surface stabilizer is a PEG-derivatized lipid, although most materials that are useful as surface stabilizers (see the auxiliary surface stabilizer section below) can be modified with PEG. In addition, many surface stabilizers described herein contain PEG chains,, such as pluronics. Suitable PEG-derivatized lipid surface stabilizers include, but are not limited to, a PEG-derivatized phospholipid, PEG-derivatized cholesterol, PEG-derivatized cholesterol derivative, PEG-derivatized vitamin A, and PEG-derivatized vitamin E.

The molecular weight of the PEG substituent on the surface stabilizer affects the circulation half life of the compound. Derivatized surface stabilizers having a PEG of high molecular mass, such as about 4000 to about 5000 Da, have long circulation half lives, with lower molecular weights of 2000 Da also being useful. Derivatized surface stabilizers having lower PEG molecular masses, such as about 750 to about 800 Da are also useful, although the circulation half-life begins to be compromised at this lower molecular weight. See e.g., Allen, “Long-circulating (sterically stabilized) liposomes for targeted drug delivery,” TiPS, 15:215-220, 218 (1994); and Yuda et al., “Prolongation of Liposome Circulation Time by Various Derivatives of Polyethyleneglycols,” Biol. Pharm. Bull., 19:1347-1351, 1347-1348, 1349 (1996).

Liposomes containing PEG-derivatives and having functional groups at their terminals, such as DPP-PEG-OH and DSPE-PEG-COOH (e.g., α-(dipalmitoylphosphatidyl)-ω-hydroxypolyoxyethylene and distearoylphosphatidyl-N-(3-carboxypropionylpolyoxyethylene succinyl)ethanolamine), also lengthen the circulation half-life of the compounds as compared to non-PEG derivatized compounds and PEG-derivatized compounds of the same molecular weight lacking the functional end group. Yuda et al. at 1349. Moreover, PEG-derivatized compounds having terminal end functional groups and lower molecular weights, e.g., about 1000 Da or less, result in longer circulation times as compared to non-PEG derivatized compounds and PEG-derivatized compounds of the same molecular weight lacking the functional end group

Two exemplary commercially available PEG-liposomes, useful as surface stabilizers in the invention, are PEG-5000™ and PEG-2000™. (Nektar Therapeutics, Inc.).

2. Secondary or Auxiliary Surface Stabilizers

The compositions of the invention can also include one or more auxiliary non-PEG-derivatized surface stabilizers in addition to the at least one PEG-derivatized surface stabilizer.

The auxiliary surface stabilizers of the invention are preferably adsorbed on, or associated with, the surface of the active agent particles. The auxiliary surface stabilizers especially useful herein preferably do not chemically react with the active agent particles or itself. Preferably, individual molecules of the auxiliary surface stabilizer are essentially free of intermolecular cross-linkages.

Two or more auxiliary surface stabilizers can be employed in the compositions and methods of the invention.

Suitable auxiliary surface stabilizers can preferably be selected from known organic and inorganic pharmaceutical excipients. Such excipients include various polymers, low molecular weight oligomers, natural products, and surfactants. Preferred surface stabilizers include nonionic and ionic surfactants. Two or more surface auxiliary stabilizers can be used in combination.

Suitable surface stabilizers can preferably be selected from known organic and inorganic pharmaceutical excipients. Such excipients include various polymers, low molecular weight oligomers, natural products, and surfactants. Preferred auxiliary surface stabilizers include nonionic, cationic, zwitterionic, and ionic compounds.

Representative examples of surface stabilizers include gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropyl celluloses (e.g., HPC, HPC-SL, and HPC-L), hydroxypropyl methylcellulose (HPMC), hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g. Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (DOSS) (American Cyanamid)); Duponol P®, which is a sodium lauryl sulfate (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G® (Olin Chemicals, Stamford, Conn.); Crodestas SL-40® (Croda, Inc.); and SA9OHCO, which is C₁₈H₃₇CH₂C(O)N(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, random copolymers of vinyl pyrrolidone and vinyl acetate, and the like.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press; 1986), specifically incorporated by reference. The surface stabilizers are commercially available and/or can be prepared by techniques known in the art.

Examples of useful cationic surface stabilizers include but are not limited to polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, a charged phospholipid such as dimyristoyl phophatidyl glycerol, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quartemary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, dodecyl trimethyl ammonium bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336198 ), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Particularly preferred nonpolymeric primary stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an immonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

D. Labeling of Active Agents, Surface Stabilizers, or Antibodies or Fragments Thereof

The active agent, surface stabilizer, or antibody or fragment thereof of the invention may be labeled with a detectable signal. Such labeling may be particularly preferable when the compositions of the invention are utilized for disease sensing or imaging.

Such labeling is accomplished by joining, either covalently or non-covalently, a substance which provides for a detectable signal with the active agent, surface stabilizer, or antibody or fragment thereof. A wide variety of labels and conjugation techniques are known and are reported extensively in both the scientific and patent literature. Suitable labels include, for example, radionucleotides, enzymes, substrates, cofactors, inhibitors, fluorescent moieties, chemiluminescent moieties, magnetic particles, and the like.

The means by which the active agent, surface stabilizer, or antibody or fragment thereof of the present invention are labeled is not a critical aspect of the invention and can be accomplished by any number of methods currently known or later developed.

Detectable labels suitable for use in the present invention include any composition detectable by suitable means, including for example, spectroscopic, radioisotopic, photochemical, biochemical, immunochemical, electrical, optical or chemical means. Useful labels in the present invention include, but are not limited to, biotin for staining with labeled streptavidin conjugate, magnetic beads, fluorescent dyes (e.g., fluorescein, Texas red, rhodamine, green fluorescent protein, and the like), radiolabels (e.g., ³H, 125I, ³⁵S, ¹⁴C, or ³²P), enzymes (e.g., horse radish peroxidase, alkaline phosphatase, and others commonly used in an ELISA), fluorophores, chemiluminescent agents, and colorimetric labels such as colloidal gold or colored glass or plastic (e.g., polystyrene, polypropylene, latex, etc.) beads. The choice of label depends on sensitivity required, stability requirements, available instrumentation, and ease of conjugation with the active agent, surface stabilizer, or antibody or fragment thereof.

Non-radioactive probes are often labeled by indirect means. For example, a lgand molecule can be covalently bound to the active agent, surface stabilizer, or antibody or fragment thereof. The ligand then binds to an anti-ligand molecule which is either inherently detectable or covalently bound to a detectable signal system, such as an enzyme, a fluorophore, or a chemiluminescent compound. Enzymes of interest as labels will primarily be hydrolases, such as phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescers include luciferin and 2,3-dihydrophthalazinediones, e.g., luminol. Ligands and anti-ligands may be varied widely. Where a ligand has a natural anti-ligand, namely ligands such as biotin, thyroxine, and cortisol, it can be used in conjunction with its labeled, naturally occurring anti-ligands. Alternatively, any haptenic or antigenic compound can be used in combination with an antibody.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

Next to immunoprecipitation and X-ray crystallography, another important approach to understanding antibody binding phenomena has been through the use of luminescent probes as model antibody ligands. The use of luminescence as a means of exploring antibody related phenomena has certain advantages. If an appropriately chosen luminescent molecule is used, the chemical composition and molecular recognition properties of an antibody binding site can be determined. In addition, luminescence is an effective spectroscopic signal by which association constants and kinetic binding parameters can be quantified.

A variety of antibodies specific to, or elicited by, luminescent molecules are known. The most extensively studied set of data for a luminescent antibody probe exists for fluorescein. The extensive number of studies that exists for this system has made anti-fluorescein antibodies a-paradigm for antibody binding in general. Both polyclonal and monoclonal antibodies have been elicited via immunization with protein conjugates prepared with fluorescein isothiocyanate. Antibody affinities have been reported to range from 10⁴ to >10¹² M⁻¹. In general, antibody binding of fluorescein results in quenching of up to 90% of the fluorescence relative to that of fluorescein in aqueous solution.

E. Nanoparticulate Active Agent Particle Size

The compositions of the invention comprise nanoparticulate active agent particles which have an effective average particle size of less than about 2 microns (i.e., 2000 nm). In preferred embodiments of the invention, the nanoparticulate active agent particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

By “an effective average particle size of less than about 2 microns” it is meant that at least 50% of the active agent particles have a particle size less than the effective average, by weight, i.e., less than about 2 microns, 1900 nm, 1800 nm, etc., when measured by the above-noted techniques. Preferably, at least about 70%, at least about 90%, at least about 95%, or at least about 99% of the active agent particles have an effective average particle size less than the effective average, i.e., less than about 2 microns, 1900 nm, 1800 nm, 1700 nm, etc.

In the present invention, the value for D50 of a nanoparticulate active agent composition is the particle size below which 50% of the active agent particles fall, by weight. Similarly, D90 is the particle size below which 90% of the active agent particles fall, by weight.

F. Concentration of Nanoparticulate Active Agent and PEG Derivatized Surface Stabilizer

The relative amount of at least one active agent and one or more PEG-derivatized surface stabilizers can vary widely. The optimal amount of the surface stabilizer can depend, for example, upon the particular active agent selected, the hydrophilic lipophilic balance (HLB), melting point, water solubility of the surface stabilizer, and the surface tension of water solutions of the stabilizer, etc.

Preferably, the concentration of the at least one active agent can vary from about 99.5% to about 0.001%, from about 95% to about 0.1%, or from about 90% to about 0.5%, by weight, based on the total combined weight of the at least one active agent and at least one PEG-derivatized surface stabilizer, not including other excipients.

The concentration of the at least one PEG-derivatized surface stabilizer can vary from about 0.5% to about 99.999%, from about 5.0% to about 99.9%, or from about 10% to about 99.5%, by weight, based on the total combined weight of the at least one active agent and at least one PEG-derivatized surface stabilizer, not including other excipients.

G. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fuimaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

III. Methods of Making Nanoparticulate Active Agent Formulations

The nanoparticulate active agent compositions can be made using, for example, milling, precipitation, or homogenization techniques. Exemplary methods of making nanoparticulate active agent compositions are described in the '684 patent. Methods of making nanoparticulate active agent compositions are also described in U.S. Pat. No. 5,518,187, for “Method of Grinding Pharmaceutical Substances;” U.S. Pat. No. 5,718,388, for “Continuous Method of Grinding Pharmaceutical Substances;” U.S. Pat. No. 5,862,999, for “Method of Grinding Pharmaceutical Substances;” U.S. Pat. No. 5,665,331, for “Co-Microprecipitation of Nanoparticulate Pharmaceutical Agents with Crystal Growth Modifiers;” U.S. Pat. No. 5,662,883, for “Co-Microprecipitation of Nanoparticulate Pharmaceutical Agents with Crystal Growth Modifiers;” U.S. Pat. No. 5,560,932, for “Microprecipitation of Nanoparticulate Pharmaceutical Agents;” U.S. Pat. No. 5,543,133, for “Process of Preparing X-Ray Contrast Compositions Containing Nanoparticles;” U.S. Pat. No. 5,534,270, for “Method of Preparing Stable Drug Nanoparticles;” U.S. Pat. No. 5,510,118, for “Process of Preparing Therapeutic Compositions Containing Nanoparticles;” and U.S. Pat. No. 5,470,583, for “Method of Preparing Nanoparticle Compositions Containing Charged Phospholipids to Reduce Aggregation,” all of which are specifically incorporated by reference.

The resultant nanoparticulate active agent dispersions can be utilized in any suitable dosage form, such as but not limited to, solid or liquid dosage formnulations, liquid dispersions, oral suspensions, gels, aerosols, ointments, creams, tablets, capsules, sachets, lozenges, powders, pills, and granules.

In addition, the resultant nanoparticulate active agent dispersions can be utilized in any suitable dosage form, such as but not limited to, controlled release formulations, fast melt formulations, lyophilized formulations, delayed release formulations, extended release formulations, pulsatile release formulations, and mixed immediate release and controlled release formulations.

A. Milling to Obtain Nanoparticulate Active Agent Dispersions

Milling of active agents to obtain a nanoparticulate active agent dispersion comprises dispersing active agent particles in a liquid dispersion media in which the active agent is poorly soluble, followed by applying mechanical means in the presence of grinding media to reduce the particle size of the active agent to the desired effective average particle size.

The active agent particles can be reduced in size in the presence of: (1) at least one antibody or a fragment thereof; (2) at least one PEG-derivatized surface stabilizer; or (3) a combination thereof, such as at least one antibody or a fragment thereof attached, directly or indirectly, to a PEG-derivatized surface stabilizer.

In a preferred embodiment, the active agent particles are reduced in size in the presence of at least one PEG-derivatized surface stabilizer, following which the antibody is attached to the surface stabilizer, either directly or indirectly.

Alternatively, either before or after attrition, the active agent particles can be contacted with: (1) at least one antibody or a fragment thereof; (2) at least one PEG-derivatized surface stabilizer; or (3) a combination thereof, such that at least one antibody or a fragment thereof is attached, either directly or indirectly, to a PEG-derivatized surface stabilizer.

Other compounds, such as a diluent, can be added to the active agent composition either before, during, or after the size reduction process. Dispersions can be manufactured continuously or in a batch mode.

B. Precipitation to Obtain Nanoparticulate Active Agent Compositions

Another method of forming the desired nanoparticulate active agent composition is by microprecipitation. This is a method of preparing stable dispersions of active agents in the presence of one or more PEG-derivatized surface stabilizers and/or at least one antibody or a fragment thereof, and one or more colloid stability enhancing surface active agents free of any trace toxic solvents or solubilized heavy metal impurities.

Such a method comprises, for example:
(1) dissolving at least one active agent in a suitable solvent;
(2) adding the formulation from step (1) to a solution comprising: (a) at least one PEG-derivatized surface stabilizer, (b) at least one antibody or a fragment thereof; or (c) a combination of (a) and (b), to form a clear solution; and (3) precipitating the formulation from step (2) using an appropriate non-solvent.

In a preferred embodiment, step (2) is conducted in the presence of at least one PEG-derivatized surface stabilizer. After particle size reduction, the antibody is then attached to the PEG-derivatized surface stabilizer, either directly or indirectly.

The method can be followed by removal of any formed salt, if present, by dialysis or diafiltration and concentration of the dispersion by conventional means. Dispersions can be manufactured continuously or in a batch mode.

C. Homogenization to Obtain Nanoparticulate Active Agent Compositions

Exemplary homogenization methods of preparing nanoparticulate active agent compositions are described in U.S. Pat. No. 5,510,118 for “Process of Preparing Therapeutic Compositions Containing Nanoparticles.” Such a method comprises dispersing active agent particles in a liquid dispersion medium, followed by subjecting the dispersion to homogenization to reduce the particle size of the active agent to the desired effective average particle size.

The active agent particles can be reduced in size in the presence of: (1) at least one antibody or a fragment thereof; (2) at least one PEG-derivatized surface stabilizer; or (3) a combination thereof (i.e., at least one antibody or a fragment thereof attached to a PEG-derivatized surface stabilizer).

In a preferred embodiment, the active agent particles are reduced in size in the presence of at least one surface stabilizer, following which the antibody is attached to the PEG-derivatized surface stabilizer, either directly or indirectly.

Alternatively, either before or after attrition, the active agent particles can be contacted with: (1) at least one antibody or a fragment thereof; (2) at least one PEG-derivatized surface stabilizer; or (3) a combination thereof (i.e., at least one antibody or a fragment thereof attached to a PEG-derivatized surface stabilizer). Other compounds, such as a diluent, can be added to the active agent composition either before, during, or after the size reduction process. Dispersions can be manufactured continuously or in a batch mode.

IV. Methods of Using the Nanoparticulate Active Agent Compositions of the Invention

The nanoparticulate active agent compositions of the invention can be used in methods of targeted delivery of the active agent. In such a method, the antibody or fragment thereof present in the composition selectively binds to a target site.

The nanoparticulate active agent compositions of the invention can be administered to a subject in any pharmaceutically acceptable manner, such as oral, pulmonary, rectal, opthalmic, colonic, parenteral (e.g., intravenous, intramuscular, or subcutaneous), intracisternal, intravaginal, intraperitoneal, local (e.g., powders, ointments, or drops), buccal, nasal, and topical administration. As used herein, the term “subject” is used to mean an animal, preferably a mammal, including a human or non-human. The terms patient and subject may be used interchangeably.

The compositions of the invention can be formulated into any pharmaceutically acceptable dosage. Exemplary solid dosage forms include, but are not limited to, liquid dispersions, liquid suspensions, gels, aerosols, ointments, creams, tablets, capsules, sachets, lozenges, powders, pills, or granules. The dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

Compositions suitable for parenteral injection may comprise physiologically acceptable sterile aqueous or nonaqueous solutions, dispersions, suspensions, emulsions, and sterile powders for reconstitution into sterile injectable solutions or dispersions. Examples of suitable aqueous and nonaqueous carriers, diluents, solvents, or vehicles including water, ethanol, polyols (propylene glycol, polyethyleneglycol, glycerol, and the like), suitable mixtures thereof, vegetable oils (such as olive oil) and injectable organic.esters such as ethyl oleate. Proper fluidity can be maintained, for example, by the use of a coating such as lecithin, by the maintenance of the required particle size in the case of dispersions, and by the use of surfactants.

The nanoparticulate active agent compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage formsfor oral administration include capsules, tablets, pills, powders, and granules. In such solid dosage forms, the nanoparticulate active agent is admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, aliginates, gelatin, polyvinylpyrrolidone, sucrose and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, dispersions, solutions, suspensions, syrups, and elixirs. In addition to the active agent, the liquid dosage forms may comprise inert diluents commonly used in the art, such as water or other solvents, solubilizing agents, and emulsifiers. Besides such inert diluents, the composition can also include adjuvants, such as wetting agents, emulsifying and suspending agents, sweetening, flavoring, and perfuming agents.

Actual dosage levels of active agent in the nanoparticulate compositions of the invention may be varied to obtain an amount of active ingredient that is effective to obtain a desired therapeutic response for a particular composition and method of administration. The selected dosage level therefore depends upon the desired therapeutic effect, the route of administration, the potency of the active agent, the desired duration of treatment, and other factors.

The daily dose may be administered in single or multiple doses. The specific dose level for any particular patient will depend upon a variety of factors including the body weight, general health, sex, diet, time and route of administration, potency of the administered active agent rates of absorption and excretion, combination with other drugs, and the severity of the particular disease being treated.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

Several of the formulations in the examples that follow were investigated using a light microscope. Here, “stable” nanoparticulate dispersions (uniform Brownian motion) were readily distinguishable from “aggregated” dispersions (relatively large, nonuniform particles without motion).

### EXAMPLE 1

This example demonstrates the preparation of a composition comprising a poorly water-soluble nanoparticulate active agent and a PEG-derivatized phospholipid surface stabilizer with a biologically-active ligand. The ligand is a biotin fimctional group covalently coupled to the terminus of the PEG chain. A binding experiment was conducted with fluorescent avidin to confirm that the activity of the biotin functional group on the surface stabilizer is maintained after particle size reduction via milling of the active agent.

The active agent used in the example was paclitaxel. Paclitaxel belongs to the group of medicines called antineoplastics. It is used to treat cancer of the ovaries, breast, certain types of lung cancer, and a cancer of the skin and mucous membranes more commonly found in patients with acquired immunodeficiency syndrome (AIDS). It may also be used to treat other kinds of cancer. Paclitaxel has the following chemical structure:

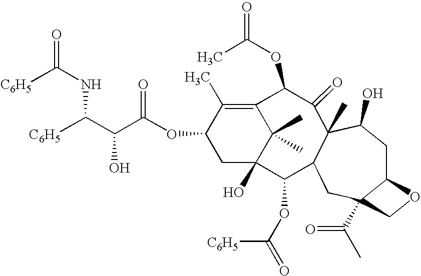

Nanoparticulate paclitaxel dispersions were prepared by milling an aqueous slurry of 5% (wt.) paclitaxel and 1.25% (wt.) PEG-derivatized phospholipid stabilizer. The preparations were ball-milled for 12-36 hours in 15 mL bottles in the presence of 0.8 mm YTZ attrition media.

Two formulations containing different PEG-derivatized phospholipids were prepared: (1) an “active” formulation contained 1,2 Dipalmitoyl-sn-Glycero-3-Phosphoethanolamine-N-[Biotinyl(Polyethylene Glycol)2000] (sodium salt), and

(2) a “control” formulation contained 1,2 Distearoyl-sn-Glycero-3-Phosphoethanolamine-N-[Methoxy(Polyethylene Glycol)2000] (both from Avanti Polar Lipids).

The particle size distributions of the milled paclitaxel formulations (Table 1) were measured by static laser light scattering using a Horiba LA910 particle size distribution analyzer (Horiba Instruments, Irvine, Calif.). The mean, D50, and D90 of the paclitaxel particles following milling is shown below.

TABLE 1
Characteristics of Milled Paclitaxel Formulations

| | Active | Surface | Particle Size (30 sec sonication) | | |
|---|---|---|---|---|---|
| Sample | Agent | Stabilizer | Mean (nm) | D50 (nm) | D90 (nm) |
| Active | Paclitaxel | Biotinylated | 175 | 163 | 241 |
| | | PEG- | | | |
| | | phospholipid | | | |
| Control | Paclitaxel | PEG- | 119 | 105 | 184 |
| | | phospholipid | | | |

A simple binding study was conducted to characterize the ability of the formulations to bind fluorescent avidin. A sample of each nanoparticulate paclitaxel formulation was washed to remove unbound PEG-derivatized phospholipid. Nanoparticulate paclitaxel particles, having the PEG-derivatized surface stabilizer associated with the surface thereof, were isolated by microcentrifugation and resuspended in deionized water.

Washed samples were incubated for I hour at room temperature with Avidin-FITC conjugate (Sigma). Free (unbound) Avidin-FITC was separated from the, nanoparticulate paclitaxel particles by microcentrifugation. The particulate fraction was isolated and dispersed in a small volume for analysis by epifluorescence and phase contrast microscopy.

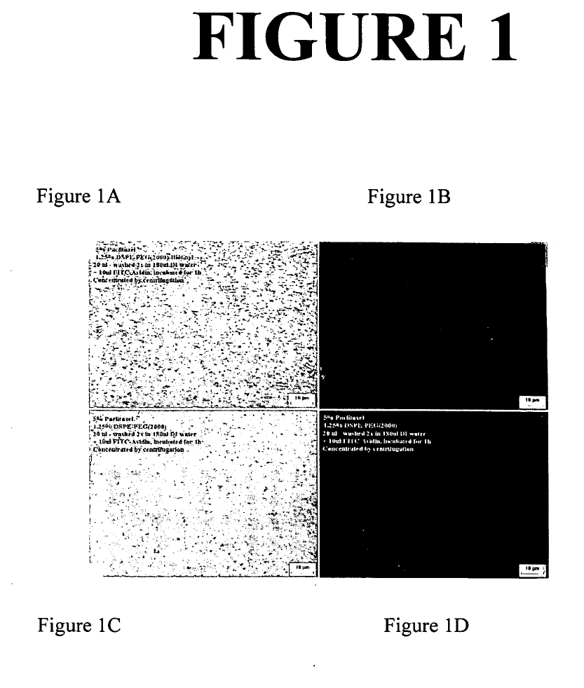

The active dispersion was highly fluorescent, indicating Avidin-FITC binding, while the control formulation displayed no detectable fluorescence (FIG. 1). This demonstrates that biotin activity of the surface stabilizer is maintained and is not lost after milling. These results suggest that PEG-derivatized phospholipids can be successfully used as nanoparticulate active agent surface stabilizers and are capable of presenting biologically-relevant ligands in an active form.

### EXAMPLE 2

This example describes the preparation of a composition comprising a poorly water-soluble nanoparticulate active agent and a PEG-derivatived phospholipid surface stabilizer with a fluorescent rhodamine label. Epifluorescence microscopy was ultilized to demonstrate that the fluorescent surface stabilizer associates with the active agent and maintains the ability to fluoresce after milling. The poorly water-soluble active agent utilized in this example was the x-ray contrast agent benzoic acid, 3,5-bis(acetylamino)-2,4,6-triodo-4-(ethyl-3-ethoxy-2-butenoate) ester (“WIN 68209”).

Two dispersions of WIN 68209 were prepared: (1) a dispersion of 5% (wt.) WIN 68209 and 0.67% PEG-derivatized 1,2-distearoyl-d62-sn-glycero-3-phosphoethanolamine (PEG-derivatized DSPE, Avanti Polar Lipids) and (2) a dispersion of 5% (wt.) WIN 68209 and 0.05% rhodamine-labeled PEG-derivatized 1,2-dipalmitoyl-d62-sn-glycero-3-phosphoethanolamine (PEG-derivatized DPPE, Avanti Polar Lipids, custom synthesis by Molecular Probes).

An aqueous slurry of 5% (wt.) WIN 68209 and 0.67% PEG-derivatized DSPE, and an aqueous slurry of % (wt.) WIN 68209 and 0.05% rhodamine-labeled PEG-derivatized DPPE, were each ball-milled for 13 hours in 15 mL bottle in the presence of 0.8 mm YTZ attrition media.

Particle size distribution of the milled WIN 68209 particles was determined by static laser light scattering in a Horiba LA910 particle size distribution analyzer (Horiba Instruments, Irvine, Calif.). The milled WIN 68209 dispersion had the following properties: mean particle size 207 nm, D50 197 nm, D90 284 nm (after 30s sonication).

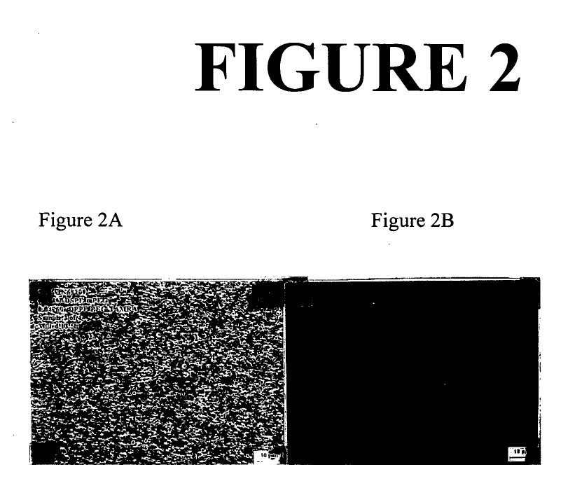

The WIN 68209 dispersion was imaged by phase contrast and epifluorescence microscopy. The WIN 68209 dispersion fluoresced brightly with epifluorescent illumination, indicating that the surface stabilizer maintained its fluorescent properties after milling (FIG. 2). This type of surface stabilizer can be useful in the fluorescent imaging and quantitation of nanoparticulate active agents with which it is associated.

### EXAMPLE 3

The purpose of this example was to demonstrate targeting of nanoparticulate active agent compositions comprising at least one antibody to cultured endothelial cells. In the following example, a biotinylated monoclonal antibody is coupled indirectly to a biotinylated PEG-derivatized surface stabilizer using the protein streptavidin as a linker.

The poorly water-soluble active agent utilized in this example was the x-ray contrast agent benzoic acid, 3,5-bis(acetylamino)-2,4,6-triodo- 4-(ethyl-3-ethoxy-2-butenoate) ester (“WIN 68209”).

Preparation of Nanoparticulate Dispersions of WIN 68209:

As described below, samples of WIN 68209 were milled in the presence of PEG-derivatized 1,2-distearoyl-d62-sn-glycero-3-phosphoethanolamine (PEG-derivatized DSPE, Avanti Polar Lipids). Targeting and fluorescence properties were conferred by including small amounts of modified versions of PEG-derivatized 1,2-dipalmitoyl-d62-sn-glycero-3-phosphoethanolamine (PEG-derivatized DPPE, Avanti Polar Lipids). Fractions of the PEG-derivatized DPPE were modified with either a fluorescent rhodamine label (DPPE-PEG-Rhodamine, Custom Synthesis, Molecular Probes) or a biotin functional group attached to the PEG chain (DPPE-PEG-Biotin, Avanti Polar Lipids). The DPPE-PEG-Rhodamine enables tracking of the compound via fluorescent detection, while the DPPE-PEG-Biotin presents an active biotin group for antibody linkage.

An aqueous slurry of 0.05 wt. % PEG-DPPE-Rhodamine, 0.05 wt. % DPPE-PEG-Biotin, 0.62% PEG-derivatized DSPE, and 5 wt. % WIN 68209, was ball milled at room temperature for 12-18 h in a 15 ml bottle in the presence of 0.8 mm YTZ milling media (active). A second sample was prepared without DPPE-PEG-Biotin, maintaining the same total amount of stabilizer (control), to be used as a negative control.

The resultant mean; D50, and D90 particle sizes of the milled dispersions of WIN 68209 was determined by static laser light scattering (Table 2) using a Horiba LA-910 Laser Scattering Particle Size Distribution Analyzer (Horiba Instruments, Irvine, Calif.).

TABLE 2
Characteristics of Milled WIN 68209 Compositions

| | | | Particle Size | | |
|---|---|---|---|---|---|
| | | | (30 s sonication) | | |
| | Active | | Mean | D50 | D90 |
| Sample | Agent | Surface Stabilizers | (nm) | (nm) | (nm) |
| Active | WIN 68209 | PEG-Derivatized DSPE | 216 | 206 | 297 |
| | | DPPE-PEG-Rhodamine | | | |
| | | DPPE-PEG-Biotin | | | |
| Control | WIN 68209 | PEG-Derivatized DSPE | 207 | 196 | 287 |
| | | DPPE-PEG-Rhodamine | | | |

Validation of Selective Binding of an Integrin-specific Antibody:

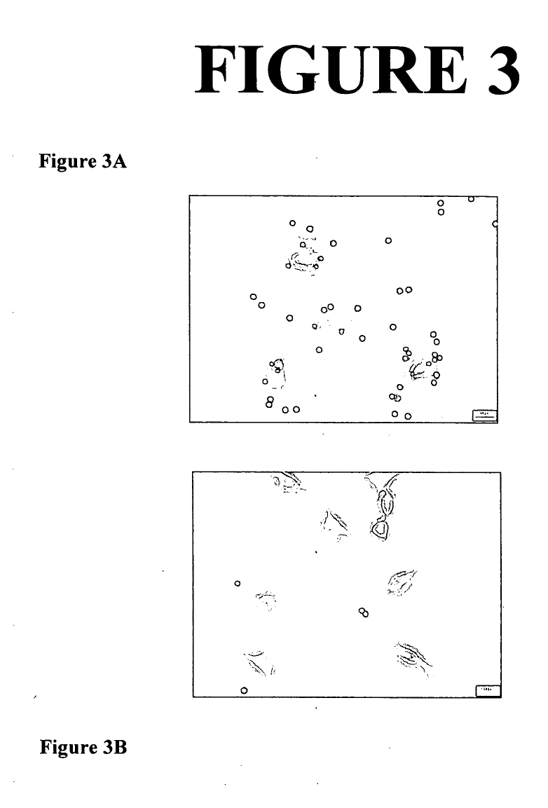

The antibody used in this example binds selectively to integrin αᵥβ₃, which is a cell-adhesion receptor expressed on endothelial cells during angiogenesis. The ability of the antibody to target WIN 68209 particles to αᵥβ₃-positive human umbilical vein endothelial cells (HUVEC, CRL-1730, American Type Culture Collection) was confirmed using streptavidin-coated fluorescent polystyrene microspheres (Fluoresbrite YG, 6 um, Polysciences). The microspheres were incubated with biotinylated antibody (CD146, Chemicon International) in phosphate buffered saline (PBS, Invitrogen) for 1 hour and then added to the cell culture. Binding was evaluated by phase-contrast and epifluorescence microscopy. The microspheres bound strongly to HUVEC but not to NIH/3T3 fibroblasts (CRL-1568, American Type Culture Collection), which do not express the receptor, thereby validating antibody selectivity (FIG. 3).

Preparation of Nanoparticulate WIN 68209 Compositions Comprising Integrin-Specific Antibody:

The milled WIN 68209 active sample was decorated with a biotinylated anti-endothelial-cell monoclonal antibody via streptavidin linkage to the biotin-functionalized stabilizer. Briefly, biotinylated antibody was precoupled to streptavidin (S0677, Sigma) at a molar ratio of 1:10 by incubating the reagents in phosphate buffered saline (PBS, Invitrogen) for 1 hour at room temperature. The nanoparticulate WIN 68209 dispersions were washed to remove soluble DPPE-PEG-Biotin, which might compete for streptavidin/antibody complexes. Dispersions were microcentrifuged to pellet the nanoparticulate WIN 68209 particles, which were isolated and resuspended in deionized water. Washed WIN 68209 nanoparticles were labeled with the streptavidin/antibody complexes by incubating for 1 hour at room temperature. Nanoparticulate WIN 68209 was used at a 50-fold weight excess to streptavidin. The control sample was treated in the same manner.

Determination of the Selective Binding Ability of Active and Control Samples:

Active and control samiples, as processed above, were incubated with (HUVEC). Binding of the WIN 68209 particles to the HUVEC was evaluated by epifluorescence and phase contrast microscopy.

Results:

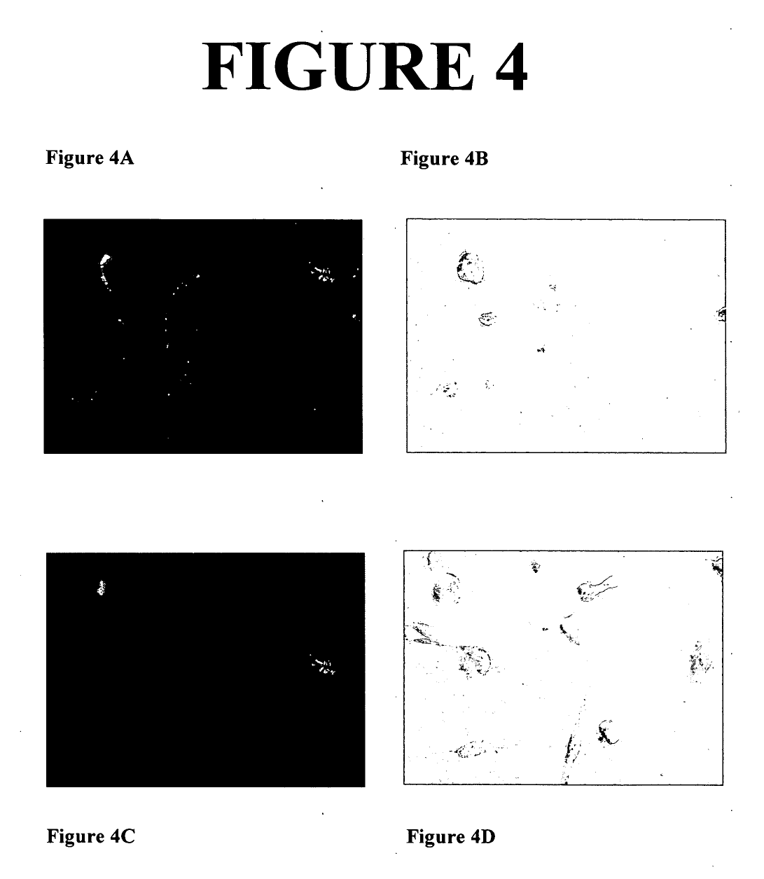

Particles of WIN 68209 present in the active sample were found to bind to HUVEC (FIG. 4). Particles of WIN 68209 present in the control sample (lacking biotinylated stabilizer) did not bind to endothelial cells with a high affinity. Removal of any component in the antibody-particle linkage (e.g. streptavidin or antibody) abolished specific binding (data not shown).

Conclusion:

This example demonstrates that the nanoparticulate WIN 68209 compositions of the invention specifically target the site of interest, based upon the binding specificity of the antibody or fragment thereof present in the composition. Thus, the compositions of the invention provide for specific targeting of poorly soluble active agents.

Throughout the specification, any and all references to a publicly available document, including a U.S. patent, are specifically incorporated by reference.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A nanoparticulate active agent composition comprising:
(a) at least one poorly soluble active agent, wherein particles of the active agent have an effective average particle size of less than about 2000 nm;
(b) associated with the surface of the active agent at least one PEG-derivatized surface stabilizer, and
(c) associated with the PEG-derivatized surface stabilizer at least one antibody or a fragment thereof, wherein the fragment has the ability to specifically bind to a target site.

2. The composition of claim 1, wherein the composition is formulated for administration selected from the group consisting of oral, pulmonary, rectal, opthalmic, colonic, parenteral, intracisternal, intravaginal, intraperitoneal, local, buccal, nasal, and topical administration.

3. The composition of claim 1 formulated into a dosage form selected from the group consisting of liquid dispersions, oral suspensions, gels, aerosols, ointments, creams, tablets, capsules, sachets, lozenges, powders, pills, and granules.

4. The composition of claim 1 formulated into a dosage form selected from the group consisting of controlled release formulations, fast melt formulations, lyophilized formulations, delayed release formulations, extended release formulations, pulsatile release formulations, and mixed immediate release and controlled release formulations.

5. The composition of claim 1, wherein the composition further comprises one or more pharmaceutically acceptable excipients, carriers, or a combination thereof.

6. The composition of claim 1, wherein the at least one active agent is present in an amount selected from the group consisting of from about 99.5% to about 0.001%, from about 95% to about 0.1%, or from about 90% to about 0.5%, by weight, based on the total combined dry weight of the at least one active agent and at least one PEG-derivatized surface stabilizer, not including other excipients.

7. The composition of claim 1, wherein the at least one surface stabilizer is present in an amount selected from the group consisting of from about 0.5% to about 99.999%, from about 5.0% to about 99.9%, or from about 10% to about 99.5%, by weight, based on the total combined dry weight of the at least one active agent and at least one PEG-derivatized surface stabilizer, not including other excipients.

8. The composition of claim 1, wherein the antibody or a fragment thereof is directly attached to the PEG-derivatized surface stabilizer.

9. The composition of claim 1, wherein the antibody or a fragment thereof is attached to the PEG-derivatized surface stabilizer via a linker.

10. The composition of claim 9, wherein the linker is a biotin fuinctional group.

11. The composition of claim 1, wherein the antibody is an IgD, IgA, IgM, IgE, or IgG immunoglobulin.

12. The composition of claim 1, wherein the antibody fragment is from an IgD, IgA, IgM, IgE, or IgG immunoglobulin.

13. The composition of claim 1, wherein the antibody fragment is selected from the group consisting of a Fab region, a F(ab′)₂ region, a Fab region, a Fab′ region, a Fv region, a VL region, a VH region, and a fragment thereof.

14. The composition of claim 1, wherein the antibody is selected from the group consisting of chimeric antibodies, humanized antibodies, and heteroconjugate antibodies.

15. The composition of claim 1, wherein the PEG-derivatized surface stabilizer is selected from the group consisting of PEG-derivatized phospholipid, PEG-derivatized cholesterol, PEG-derivatized cholesterol derivative, PEG-derivatized vitamin A, and PEG-derivatized vitamin E.

16. The composition of claim 1, comprising at least two surface stabilizers.

17. The composition of claim 16, wherein at least one surface stabilizer is selected from the group consisting of an anionic surface stabilizer, a cationic surface stabilizer, a zwitterionic surface stabilizer, and an ionic surface stabilizer.

18. The composition of claim 17, wherein at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, random copolymers of vinyl acetate and vinyl pyrrolidone, cationic surface stabilizer is selected from the group consisting of cationic polymers, cationic biopolymers, cationic polysaccharides, cationic cellulosics, cationic alginates, cationic nonpolymeric compounds, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10198 , tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

19. The composition of claim 17, wherein the composition is bioadhesive.

20. The composition of claim 18, wherein the composition is bioadhesive.

21. The composition of claim 1, comprising as a surface stabilizer PEG-derivatized DPPE.

22. The composition of claim 1, wherein the active agent is selected from the group consisting of a crystalline phase, an amorphous phase, a semi-crystalline phase, a semi-amorphous phase, and mixtures thereof.

23. The composition of claim 1, wherein the effective average particle size of the active agent particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

24. The composition of claim 1, further comprising at least one additional active agent composition having an effective average particle size which is different that the effective average particle size of the active agent composition of claim 1.

25. The composition of claim 1, wherein the active agent is selected from the group consisting of nutraceuticals, COX-2 inhibitors, retinoids, anticancer agents, NSAIDS, proteins, peptides, nucleotides, anti-obesity drugs, dietary supplements, carotenoids, corticosteroids, elastase inhibitors, anti-ftngals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytics, sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants, anoretics, sympathomimetics, thyroid agents, vasodilators, xanthines, acyclovir, alprazolam, altretamine, amiloride, amiodarone, benztropine mesylate, bupropion, cabergoline, candesartan, cerivastatin, chlorpromazine, ciprofloxacin, cisapride, clarithromycin, clonidine, clopidogrel, cyclobenzaprine, cyproheptadine, delavirdine, desmopressin, diltiazem, dipyridamole, dolasetron, enalapril maleate, enalaprilat, famotidine, felodipine, furazolidone, glipizide, irbesartan, ketoconazole, lansoprazole, loratadine, loxapine, mebendazole, mercaptopurine, milrinone lactate, minocycline, mitoxantrone, nelfinavir mesylate, nimodipine, norfloxacin, olanzapine, omeprazole, penciclovir, pimozide, tacolimus, quazepam, raloxifene, rifabutin, rifampin, risperidone, rizatriptan, saquinavir, sertraline, sildenafil, acetyl-sulfisoxazole, temazepam, thiabendazole, thioguanine, trandolapril, triamterene, trimetrexate, troglitazone, trovafloxacin, verapamil, vinblastine sulfate, mycophenolate, atovaquone, atovaquone, proguanil, ceftazidime, cefuroxime, etoposide, terbinafine, thalidomide, fluconazole, amsacrine, dacarbazine, teniposide, acetylsalicylate, and an active agent usefuil in dermal applications.

26. The composition of claim 25, wherein the nutraceutical is selected from the group consisting of dietary supplements, vitamins, minerals, herbs, lutein, folic acid, fatty acids, fruit extracts, vegetable extracts, phosphatidylserine, lipoic acid, melatonin, glucosamine/chondroitin, Aloe Vera, Guggul, glutamine, amino acids, green tea, lycopene, whole foods, food additives, herbs, phytonutrients, antioxidants, flavonoid constituents of fruits, evening primrose oil, flax seeds, fish oils, marine animal oils, and probiotics.

27. The composition of claim 25, wherein the anticancer agent is selected from the group consisting of alkylating agents, antimetabolites, anthracenediones, natural products, hormones, antagonists, radiosensitizers, platinum coordination complexes, adrenocortical suppressants, immunosuppressive agent, substituted ureas, COX-2 inhibitors, cisplatin, carboplatin, mitoxantrone, hydroxyurea, mitotane, aminoglutethimide, cyclosporine, azathioprine, sulfasalazine, methoxsalen, and thalidomide.

28. The composition of claim 27, wherein:
(a) the alkylating agent is selected from the group consisting of chlormethine, chlorambucile, melphaian, uramustine, mannomustine, extramustinephoshate, mechlore-thaminoxide, cyclophosphamide, ifosfamide, trifosfamide, tretamine, thiotepa, triaziquone, mitomycine, busulfan, piposulfan, piposulfam, carmustine, lomustine, semustine, streptozotocine, mitobronitole, dacarbazine and procarbazine; or
(b) the antimetabolite is selected from the group consisting of methotrexate, fluorouracil, floxuridine, tegafur, cytarabine, idoxuridine, flucytosine, mercaptopurine, thioguanine, azathioprine, tiamiprine, vidarabine, pentostatin, and puromycine; or
(c) the natural product is selected from the group consisting of vinblastine, vincristine, etoposide, teniposide, adriamycine, daunomycine, doctinomycin, daunorubicin, doxorubicin, mithramycin, bleomycin, mitomycin, L-asparaginase, alpha-interferon, camptothecin, taxol, and retinoic acid; or
(d) the hormone or antagonist is selected from the group consisting of prednisone, hydroxyprogesterone caproate, medroxyprogesterone acetate, megestrol acetate, diethylstilbestrol, ethinyl estradiol, tamoxifen, testosterone propionate, fluoxymesterone, flutamide, and leuprolide.

29. The composition of claim 25, wherein the NSAID is selected from the group consisting of nabumetone, tiaramide, proquazone, bufexamac, flumizole, epirazole, tinoridine, timegadine, dapsone, aspirin, diflunisal, benorylate, fosfosal, diclofenac, alclofenac, fenclofenac, etodolac, indomethacin, sulindac, tolmetin, fentiazac, tilomisole, carprofen, fenbufen, flurbiprofen, ketoprofen, oxaprozin, suprofen, tiaprofenic acid, ibuprofen, naproxen, fenoprofen, indoprofen, pirprofen, flufenamic, mefenamic, meclofenamic, niflumic, oxyphenbutazone, phenylbutazone, apazone, feprazone, piroxicam, sudoxicam, isoxicam, and tenoxicam.

30. The composition of claim 25, wherein the COX-2 inhibitor is selected from the group consisting of nimesulide, celecoxib, rofecoxib, meloxicam, valdecoxib, parecoxib, etoricoxib, flurbiprofen, nabumetone, etodolac, iguratimod, flosulide, piroxicam, diclofenac, lumiracoxib, monteleukast, pranlukast, heptinylsulfide, SC-236, SC-58125, SC-57666, SC-558, SC-560, SC 41930, NS-398, DFU, L-745337, L-761066, L-761000, L-748780, DUP-697, PGV 20229, BF 389, CL 1004, PD 136005, PD 142893, PD 138387, PD 145065, D 1367, R 807, JTE-522, FK-3311, FK 867, FR 140423, FR 115068, GR 253035, RWJ 63556, RWJ 20485, ZK 38997, S 2474, RS 57067, RS 104897, RS 104894, and SB 209670.

31. The composition of claim 1, wherein upon administration to a mammal the active agent particles redisperse such that the particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 mn, less than about 75 nm, and less than about 50 nm.

32. The composition of claim 1, wherein the composition redisperses in a biorelevant media such that the active agent particles have an effective average particle size selected from the group consisting of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm

33. The composition of claim 32, wherein the biorelevant media is selected from the group consisting of water, aqueous electrolyte solutions, aqueous solutions of a salt, aqueous solutions of an acid, aqueous solutions of a base, and combinations thereof.

34. The composition of claim 1, wherein the Tₘₐₓ of the active agent, when assayed in the plasma of a mammalian subject following administration, is less than the Tₘₐₓ for a non-nanoparticulate composition of the same active agent, administered at the same dosage.

35. The composition of claim 34, wherein the Tₘₐₓ is selected from the group consisting of not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, and not greater than about 5% of the Tₘₐₓ exhibited by a non-nanoparticulate composition of the same active agent, administered at the same dosage.

36. The composition of claim 1, wherein the Cₘₐₓ of the active agent, when assayed in the plasma of a mammalian subject following administration, is greater than the Cₘₐₓ for a non-nanoparticulate composition of the same active agent, administered at the same dosage.

37. The composition of claim 36, wherein the Cₘₐₓ is selected from the group consisting of at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by a non-nanoparticulate composition of the same active agent, administered at the same dosage.

38. The composition of claim 1, wherein the AUC of the active agent, when assayed in the plasma of a mammalian subject following administration, is greater than the AUC for a non-nanoparticulate composition of the same active agent, administered at the same dosage.

39. The composition of claim 38, wherein the AUC is selected from the group consisting of at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate formulation of the same active agent, administered at the same dosage.

40. The composition of claim 1 which does not produce significantly different absorption levels when administered under fed as compared to fasting conditions.

41. The composition of claim 40, wherein the difference in absorption of the active agent composition of the invention, when administered in the fed versus the fasted state, is selected from the group consisting of less than about 100%, less than about 90%, less than about 80%, less than about 70%, less than about 60%, less than about 50%, less than about 40%, less than about 30%, less than about 25%, less than about 20%, less than about 15%, less than about 10%, less than about 5%, and less than about 3%.

42. The composition of claim 1, wherein administration of the composition to a human in a fasted state is bioequivalent to administration of the composition to a subject in a fed state.

43. The composition of claim 42, wherein “bioequivalency” is established by:
(a) a 90% Confidence Interval of between 0.80 and 1.25 for both Cₘₐₓ and AUC; or (b) a 90% Confidence Interval of between 0.80 and 1.25 for AUC and a 90% Confidence Interval of between 0.70 to 1.43 for Cₘₐₓ.

44. The composition of claim 1 formulated into a liquid dosage form, wherein the dosage form has a viscosity, measured at 20° C., at a shear rate of 0.1 (1/s), selected from the group consisting of less than about 2000 mpa.s, from about 2000 mpa.s to about 1 mPa.s, from about 1900 mPa.s to about 1 mpa.s, from about 1800 mPa.s to about 1 mpa.s, from about 1700 mPa.s to about 1 mPa.s, from about 1600 mPa.s to about 1 mpa.s, from about 1500 mPa.s to about 1 mPa.s, from about 1400 mPa.s to about 1 mPa.s, from about 1300 mPa.s to about 1 mPa.s, from about 1200 mPa.s to about 1 mPa.s, from about 1100 mPa.s to about 1 mPa.s, from about 1000 mPa.s to about 1 mPa.s, from about 900 mPa.s to about 1 mPa.s, from about 800 mPa.s to about 1 mPa.s, from about 700 mPa.s to about 1 mPa.s, from about 600 mPa.s to about 1 mPa.s, from about 500 mPa.s to about 1 mPa.s, from about 400 mPa.s to about 1 mPa.s, from about 300 mPa.s to about 1 mPa.s, from about 200 mPa.s to about 1 mPa.s, from about 175 mPa.s to about 1 mPa.s, from about 150 mPa.s to about 1 mPa.s, from about 125 mPa.s to about 1 mPa.s, from about 100 mPa.s to about 1 mPa.s, from about 75 mPa.s to about 1 mPa.s, from about 50 mPa.s to about 1 mPa.s, from about 25 mPa.s to about 1 mPa.s, from about 15 mPa.s to about 1 mPa.s, from about 10 mPa.s to about 1 mPa.s, and from about 5 mPa.s to about 1 mPa.s.

45. The composition of claim 44, wherein the viscosity of the dosage form is selected from the group consisting of less than about 1/200, less than about 1/100, less than about 1/50, less than about 1/25, and less than about 1/10 of the viscosity of a liquid dosage form of a non-nanoparticulate composition of the same active agent, at about the same concentration per ml of active agent.

46. The composition of claim 44, wherein the viscosity of the dosage form is selected from the group consisting of less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, and less than about 90% of the viscosity of a liquid dosage form of a non-nanoparticulate composition of the same active agent, at about the same concentration per ml of active agent.

47. A method of making an active agent composition comprising:
(a) contacting particles of active agent with at least one PEG-derivatized surface stabilizer for a time and under conditions sufficient to provide an active agent composition having an effective average particle size of less than about 2000 nm; and
(b) contacting the active agent/PEG-derivatized surface stabilizer composition with at least one antibody or a fragment thereof, wherein the fragment has the ability to specifically bind to a target site, such that the antibody or a fragment thereof is associated with the PEG-derivatized surface stabilizer.

48. A method of treating a subject in need comprising administering to the subject an effective amount of a composition comprising:
(a) at least one poorly soluble active agent, wherein particles of the active agent have an effective average particle size of less than about 2000 nm;
(b) associated with the surface of the active agent at least one PEG-derivatized surface stabilizer, and
(c) associated with the PEG-derivatized surface stabilizer at least one antibody or a fragment thereof, wherein the fragment has the ability to specifically bind to a target site.
